SEQUENCE LISTING (13 sequences)

SEQ ID NO: 1 - protein, 20 aa, human
MEAADASRSNGSSPEARDAR

SEQ ID NO: 2 - protein, 17 aa, human
ELHLKPHLEGAAFRDHQ

SEQ ID NO: 3 - protein, 18 aa, human
EGEGLGQSLGNFKDDLLN

SEQ ID NO: 4 - protein, 17 aa, human
RETIPAKLVQSTLSDLR

SEQ ID NO: 5 - protein, 17 aa, human
DPAKVQSLVDTIREDPD

SEQ ID NO: 6 - protein, 15 aa, human
RVAAKRLKEGDTMMG

SEQ ID NO: 7 - protein, 19 aa, human
KTVESLEETLKKASPDGYD

SEQ ID NO: 8 - protein, 17 aa, human
TTHSISDGKDLEKLLTE

SEQ ID NO: 9 - protein, 24 aa, human
EYHKVHQMMREQSILSPSPYEGYR

SEQ ID NO: 10 - protein, 23 aa, human
RHQLLCFKEDCQAVFQDLEGVEK

SEQ ID NO: 11 - protein, 19 aa, human
DLEVKDWMQKKRRGLRNSR

SEQ ID NO: 12 - DNA, 30 bp, human
ggatccatggaagctgcagatgcctccagg

SEQ ID NO: 13 - DNA, 71 bp, human
accggtgtgcatcctccgccgccgcatcctccgccgccggcggctggggcttcgttggacccaatcccgtt

(19) United States
(12) Patent Application Publication
(10) Pub. No.: US 2007/0298444 A1
(43) Pub. Date: Dec. 27, 2007

(54) Methods and Compositions for Use in Evaluating and Treating Neoplastic Disease Conditions

(76) Inventor: Mitchell Ehren Garber, Stanford, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/594,940

(22) PCT Filed: Mar. 24, 2005
(86) PCT No.: PCT/US05/09926

§ 371 Date: Jun. 25, 2007

Related U.S. Application Data
(60) Provisional application No. 60/558,953, filed on Apr. 2, 2004.
(60) Provisional application No. 60/629,527, filed on Nov. 18, 2004.

(51) Int. Cl. G01N 33/574

(52) U.S. Cl. 435/7.23

(57) ABSTRACT

Methods and compositions for use in a evaluating and treating neoplastic disease conditions are provided. In certain embodiments of the subject invention, the presence of at least one target protein associated with cellular locomotion is determined in a cell to make an evaluation regarding the cell and/or host from which the cell was obtained. In yet other embodiments, the activity certain embodiments, the target protein is part of a nucleus-associated ribbon-like structure. Also provided are kits and pharmaceutical compositions that find use in various embodiments invention finds use in a variety of different applications, including both diagnostic and therapeutic applications.

### CROSS-REFERENCE TO RELATED APPLICATIONS

Pursuant to 35 U.S.C. § 119 (e), this application claims priority to the filing date of the U.S. Provisional Patent Application Ser. No. 60/629,527 filed Nov. 18, 2004 and to the filing date of U.S. Provisional Patent Application Ser. No. 60/558,953 filed on Apr. 2, 2004; the disclosures of which are herein incorporated by reference.

### INTRODUCTION

### Background of the Invention

Cancer is the second leading cause of death in the United States. In 1999 there were an estimated 563,100 cancer deaths and each year about 1,222,000 new cancer cases are diagnosed. Among these, solid tumor cancers such as lung, breast, prostate and colorectal cancers are the most common.

Lung cancer is the leading cause of cancer death in both men and women in Western society. If lung cancer is found and treated early, before it has spread to lymph nodes or other organs, the five-year survival rate is about 42%. However, few lung cancers are found at this early stage. Since most people with early lung cancer do not have any symptoms, only about 15% of lung cancers are found in the early stages. There are two major types of lung cancer. The first is non-small cell lung cancer. The other is small cell lung cancer. If the cancer has features of both types, it is called mixed small cell/non-small cell cancer.

Non-small cell lung cancer (NSCLC) is the most common type of lung cancer, accounting for almost 80% of lung cancers. Risk factors for NSCLC include prior smoking, passive smoking, and radon exposure. The main types of NSCLC are squamous cell carcinoma, adenocarcinoma, bronchoalveolar carcinoma, large cell carcinoma, adenosquamous carcinoma, and undifferentiated carcinoma. Squamous cell carcinoma forms in cells lining the airways. Adenocarcinoma is the most common type of non-small cell lung cancer and is the form that often occurs in people who have never smoked.

Lung cancer is best treated when it is diagnosed early. However, most patients are not diagnosed until they exhibit symptoms. Symptoms of lung cancer include cough or chest pain, a wheezing sound when breathing, shortness of breath, coughing up blood, hoarseness, or swelling in the face and neck. When a patient exhibits symptoms of lung cancer, a bronchoscopy is performed so that cells from the walls of the bronchial tubes may be examined and small pieces of tissue removed for biopsy. If the suspect tissue is unable to be obtained through this method, needle aspiration biopsy may be performed in which a needle inserted between the ribs to draw cells from the lung, or surgery is performed to remove tissue for biopsy. Diagnosis of cancer is made by examination of the characteristics of the cells under a microscope.

The following stages are used for classifying lung cancer:

Occult stage: Cancer cells are found in sputum, but no tumor can be found in the lung.

Stage 0: Cancer is only found in a local area and only in a few layers of cells. It has not grown through the top lining of the lung. Another term for this type of cell lung cancer is carcinoma in situ.

Stages I & II For a description, see a standard textbook in the field, e.g., DeVita et al., Principles and Practices of Oncology, 5.sup.th Edition, Lippincolt-Ravey, pp. 858-911

Stage III: Cancer has spread to the chest wall or diaphragm near the lung; or the cancer has spread to the lymph nodes in the area that separates the two lungs (mediastinum); or to the lymph nodes on the other side of the chest or in the neck. Stage III is further divided into stage IIIA (usually may be operated upon) and stage IIIB (usually may not be operated on).

Stage IV: Cancer has spread to other parts of the body.

Recurrent: Cancer has come back (recurred) after previous treatment.

Treatment for lung cancer depends on the stage of the disease, the age of the patient, and the overall condition of the patient. Patients may be divided into three groups, depending on the stage of the cancer and the treatment that is planned. The first group (stages 0, I, and II) includes patients whose cancers can be taken out by surgery. The second group (stage III) of patients has lung cancer that has spread to nearby tissue or to mediastinal or supraclavicular lymph nodes. These patients may be treated with radiation therapy alone or with surgery and radiation, chemotherapy and radiation, or chemotherapy alone. The group of patients with most advanced lung cancers (stage IV) are generally treated with chemotherapy alone, or a combination of chemotherapy and radiation therapy. Surgery generally is not a treatment option for Stage IV lung cancer. The most effective treatment is chemotherapy, either alone or in combination with radiation therapy. The exact treatment depends on the extent of the cancer (limited or extensive stage).

There is a need in the art for improved methods for detecting and treating cancers, including lung cancers.

Relevant Literature

Of interest are U.S. Pat. Nos. 6,667,154 and 6,509,316, as well as published application nos. 20030219768; 20030236209; 20020192228 and 20020035060. Also of interest are: Garber et al., Proc. Nat'l Acad. Sci. USA (2001) 98:13784-13789; Troyanskaya et al., Bioinformatics. (2002) 18:1454-61.

### SUMMARY OF THE INVENTION

Methods and compositions for use in a evaluating and treating neoplastic disease conditions are provided. In certain embodiments of the subject invention, the presence of at least one target protein associated with cellular locomotion, e.g., a nucleus-associated ribbon-like structure protein, is determined in a cell to make an evaluation regarding the cell and/or host from which the cell was obtained. In yet other embodiments, the activity of at least one target protein associated with cellular locomotion, e.g., nucleus-associated ribbon-like structure protein is modulated, e.g., inhibited. In certain embodiments, the target protein is present in a nucleus-associated ribbon-like structure. Also provided are kits and pharmaceutical compositions that find use in various embodiments of the subject invention. The invention finds use in a variety of different applications, including both diagnostic and therapeutic applications.

### BRIEF DESCRIPTION OF THE FIGURES

FIG. 1. cDNA microarray analysis showed characteristic gene expression patterns for five genes across two lung cancer datasets. Gene expression patterns for both the adenocarcinoma (adeno) and squamous tumor datasets were visualized in TreeView. A) Expression of 5 genes across 6 normal lung tissues, indicated by the asterisk, and 35 adenocarcinomas of the lung. The definition of adeno groups 1-3, as well as raw data files for all lung adenocarcinomas, was described previously [Garber, Proc Natl Acad Sci USA. (2001), 98(24):13784-9], (http://genome-www.stanford.edu/lung_cancer/adeno). Group 3 lung adenocarcinomas were relatively poor prognosis. The data for each gene was median centered. B) Expression of 5 genes across 3 normal lung tissues and 67 lung and head/neck squamous tumors (see FIGS. 12 and 13 below). An asterisk denotes normal lung tissue samples. The data for each gene was median centered.

FIG. 2. Peptide affinity-purified polyclonal antisera to five proteins were used for western blot analysis. Protein lysates were prepared from both D51 (adeno) and HBEC cell cultures, as indicated above the lane. Proteins (30 μg lysate) were separated on 4-20% gradient SDS-PAGE and blots were probed with polyclonal antisera to NTRK2/TrkB (lanes 1,2), Hs.516830 (RLA1) (lanes 3,4), TRIM29 (lanes 5,6), OKL38 (lanes 7,8), and LTB4DH (lanes 9,10). Molecular weight standards are shown.

FIG. 3. Immunofluorescence microscopy localized LTB4DH to a ribbon-like structure in primary human bronchial epithelial cell cultures. A) Double-label experiments to LTB4DH (green, FITC) (right), alpha-tubulin (red, rhodamine) (center, and DNA (blue, DAPI). Three channels were merged (left). Images were taken on DeltaVision (60×) followed by deconvolution. B) LTB4DH (green) localized to a ribbon-like structure that spanned the length of the nucleus (blue). Conditions were identical to 3A). C) LTB4DH (green) localized to a ribbon-like structure that sits in a nuclear groove. Conditions were identical to 3A).

FIG. 4. Hs.516830 localized to a ribbon-like structure in primary human bronchial epithelial cell cultures using immunofluorescence microscopy. Double-label experiments to Hs.516830 (green, FITC) and alpha-tubulin (red, rhodamine). DNA was stained with DAPI (blue). Sections (0.8 micron) from the bottom (left) and top (right) of the cell were optically separated using DeltaVision software.

FIG. 5. Immunofluorescence microscopy localized OKL38 protein to a ribbon-like structure in primary human bronchial epithelial cell cultures. A) Double-label experiments to OKL38 (green, FITC) and alpha-tubulin (red, rhodamine) co-localized the proteins to the mitotic spindle of a dividing cell. The cell culture was asynchronous. DNA was stained with DAPI (blue). B) Double-label experiments to OKL38 and alpha-tubulin in an interphase cell. Optical sections from top to bottom of the cell (50 planes at 0.2 microns each) were deconvolved and volume rendered using DeltaVision software. C) The cell in B) was optically sliced through the nucleus, perpendicular to the ribbon-like structure, and the slice was rotated toward the viewer using Volocity software. The still frame shown in C) was taken from the movie. D) Double-label experiments to OKL38 (left) and alpha-tubulin (right). OKL38 localized to a ribbon-like structure with a tripod orientation. E) Optical sections from D) (50 planes at 0.2 microns each) were volume rendered, optically sliced, and rotated toward the viewer as described in B).

FIG. 6. The ribbon-like structure was observed in cells differentially sensitive to nocodazole. Primary human bronchial epithelial cell cultures were treated with nocodazole and allowed to recover in drug-free medium as described in Methods. The timeline for nocodazole treatment is shown at the top of the figure. Using immunofluorescence microscopy and double-label experiments to alpha-tubulin (red, rhodamine), microtubules re-polymerized within a 20 minute recovery from nocodazole treatment (left). A cell that failed to re-polymerize microtubules within the 20 minute recovery localized Hs.516830 (green, FITC) to a ribbon-like structure (right). DNA was stained with DAPI (blue). Two channels, including DAPI, were merged (center).

FIG. 7. Using immunofluorescence microscopy, TRIM29 and NTRK2/TrkB proteins localized to primary human bronchial epithelial cells that were differentially sensitive to nocodazole. A) Triple-label experiments to filamentous actin (green, phalloidin-rhodamine) and alpha-tubulin (red, Cy5). Cells were treated with nocodazole and allowed to recover as described in Methods. A single cell in the field of view failed to re-polymerize microtubules within the 20 minute recovery from nocodazole (center cell). DNA was stained with DAPI (blue). B) The same cell in A) that failed to re-polymerize microtubules within the 20 minute recovery localized TRIM29 (green, FITC) to a ribbon-like structure. Filamentous actin (phalloidin-rhodamine) is now shown in red. C) Triple-label experiments to filamentous actin (green, phalloidin-rhodamine) and alpha-tubulin (red, Cy5), as indicated in A). D) The same cell shown in C) that failed to re-polymerize microtubules within the 20 minute recovery localized NTRK2/TrkB (green, FITC) to vesicles in the vicinity of an actin protrusion. Filamentous actin (phalloidin-rhodamine) is now shown in red.

FIG. 8. TRIM29 fused to GFP localized specifically to cellular structures in primary HBEC cultures, unless indicated otherwise. Cells were infected with a retrovirus expressing TRIM29 fused to GFP (see Methods). TRIM29 (GFP, green), actin (red, phalloidin-rhodamine), and DNA (blue, DAPI) were visualized by fluorescence microscopy (60× objective, DeltaVision). Images were deconvolved unless specified. A) TRIM29-GFP localized to multiple structures very similar to the size of a centriole/basal body. The image represents a single optical plane (0.2 microns) and was not deconvolved. B) TRIM29-GFP localized to a ribbon-like structure. Stacked images (2.2 microns) were taken from the top half of the cell. C-E) TRIM29-GFP localized to one of two nuclei in a bi-nucleated cell. Six optical sections (1.2 microns) were taken from both the bottom (C) and top (D, E) of the same cell. Images shown in D) and E) were taken from the top of the cell shown in C) with an expanded view of the nucleus and ribbon structure and with (D) or without (E) the red channel representing the actin filaments. F) TRIM29-GFP localized to ribbon-like structures in the human cell line 293T. 48 optical sections (0.2 microns each) were stacked in volume view.

FIG. 9. Confocal microscopy and live cell images localized TRIM29-GFP to a ribbon-like structure in a migrating bronchial epithelial cell. The movie sequence represents 16 time points that spanned 1.5 hours. Each time point is a compilation of 26 optical planes (0.45 microns) stacked in volume view. Still frames representing time points 1, 3, 5, and 7 are shown in A). Still frame #1 was rendered in 3D and the ribbon-like structures were rotated in virtual space. Two different orientations of the rendered object are shown in B).

FIG. 10. Gene expression patterns segregate human squamous tumors from lung and head/neck. 40 lung (LC) and 27 head/neck (HN) squamous tumors were sorted by unsupervised hierarchical clustering. Patient identification numbers are shown below the dendogram branches. Lung SCC (blue) and head/neck (orange) were colored for simplicity. Resected tumors, including three normal lung samples (normal, black), were obtained from Charite Hospital, Berlin.

FIG. 11. Gene expression patterns associated with squamous tumors. A) 70 tissues (columns) and 1600 genes (rows) were sorted by hierarchical cluster based on a similarity of gene expression. Gene clusters were extracted in the region designated by the colored bar and expanded to the right. B, C) Squamous tumors from head/neck and lung showed differential gene expression. D) Squamous lung tumors share gene expression patterns characteristic of normal lung. Genes indicated by the arrow were selected for immunohistochemistry or in situ hybridization (see FIG. 12). Due to space restrictions, only select gene names are shown.

FIG. 12. Genes were expressed in squamous tumor cells using formalin-fixed, paraffin-embedded human tumors. The name of the gene is shown above each picture. In situ hybridization identified MGC-14128 and CAII mRNAs in SCC of the head/neck. In situ hybridization localized LTB4DH, OKL38, and SFTPA1 mRNAs in SCC of the lung. Immunohistochemistry and affinity-purified antisera localized AKR1C3, NTRK2/TrkB, and Hs.516830 protein in SCC of the lung.

FIG. 13. Using immunohistochemistry and specific antiserum, TRIM29 protein localized to several non-tumor human tissues. Formalin-fixed, paraffin-embedded tissues included colon adenoma (A) and normal appendix (B).

FIG. 14. Immunohistochemistry showed that TRIM29 protein was expressed in adenocarcinoma of the lung (A) and squamous cell carcinoma of the head/neck (B). Resected tumor specimens were formalin-fixed and paraffin-embedded.

FIG. 15. The ribbon-like structure localized in the vicinity of condensed, mitotic chromosomes in HBEC treated with paclitaxel. Images were obtained by immunofluorescence microscopy (60× objective, DeltaVision) followed by deconvolution. Specific antisera were directed against microtubules (A and B), OKL38 (B), LTB4DH (C), RLA1 (D) and TRIM29 (E), as indicated in the figure. DNA (blue) was stained with DAPI. Images represent stacked optical z-sections with the following thickness: 3.0 microns for A, B, and C, 4.2 microns for D; and 5 microns for E.

FIG. 16. The ribbon-like structure localized in the vicinity of condensed, mitotic chromosomes in MCF10A breast epithelial cells treated with paclitaxel. Images were obtained by immunofluorescence microscopy (100× objective, DeltaVision) followed by deconvolution. Specific antisera were directed against LTB4DH (A) and RLA1 (B), shown in green. DNA (blue) was stained with DAPI. Images represent 14 stacked optical z-sections (2.8 microns).

FIG. 17. TRIM29-GFP (left, green) and anti-TRIM29 (right, red) co-localized to a ribbon-like structure in MCF10A breast epithelial cells treated with paclitaxel. Images were obtained by immunofluorescence microscopy (100× objective, DeltaVision) followed by deconvolution. DNA (blue) was stained with DAPI. Images represent 13 stacked optical z-sections (2.6 microns). (A, B, C) Images from three independent cells show diversity in the ribbon-like structure.

FIG. 18. RLA1 (Hs.516830) co-localized with mitochondria in breast MCF10A cells. Mitotracker Green (left, green) (Molecular Probes) and anti-Hs.516830 (middle, red) localized to small foci using fluorescence microscopy (100× objective, DeltaVision) followed by deconvolution. The two channels were merged (right). DNA was stained with DAPI (blue).

### DESCRIPTION OF THE SPECIFIC EMBODIMENTS

Methods and compositions for use in a evaluating and treating neoplastic disease conditions are provided. In certain embodiments of the subject invention, the presence of at least one target protein associated with cellular locomotion, e.g., a nucleus-associated ribbon-like structure protein, is determined in a cell to make an evaluation regarding the cell and/or host from which the cell was obtained. In yet other embodiments, the activity of at least one target protein associated with cellular locomotion, e.g., nucleus-associated ribbon-like structure protein is modulated, e.g., inhibited. In certain embodiments, the target protein is part of a nucleus-associated ribbon-like structure. Also provided are kits and pharmaceutical compositions that find use in various embodiments of the subject invention. The invention finds use in a variety of different applications, including both diagnostic and therapeutic applications.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All patents, patent applications and publications mentioned herein are incorporated herein by reference to disclose and describe the methods and/or materials in connection with which the patents, patent applications and publications are cited. The citation of any patent, patent application and publication is for its disclosure prior to the filing date and should not be construed as an admission that the present invention is not entitled to antedate such patent, patent application and publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

In further describing the subject invention, the subject methods are described first in greater detail, followed by a review of various representative applications in which the methods find use. Finally, systems and kits that find use in practicing various aspects of the subject invention are discussed.

Methods

As summarized above, the subject invention provides various methods, which may include both diagnostic and therapeutic methods. For ease of description, each of these broad categories of methods is reviewed separately below.

Diagnostic Methods

The subject invention provides methods of determining or diagnosing the metastatic propensity of a cell. As such, the subject invention provides methods of determining whether a tumor, from which an assayed cell is obtained, is metastatic. In other words, the subject invention provides methods of determining whether cells of a given tumor will spread from the tumor to other locations in a subject.

In practicing these embodiments of the subject methods, a cellular sample, e.g., biopsy, is obtained from a host and then assayed for at least the presence, and in certain embodiments the location, of at least one nucleus-associated ribbon-like structure protein. By “assayed for the presence” is meant that the cell is screened for the presence or absence of one or more target proteins. When the assay includes determining the location of the one or more target proteins, the cell is also screened to identify the cellular location of one or more target proteins, if present. In other words, the location of a target protein or proteins is determined when the particular target protein(s) is present.

By at least one target protein is meant that a given assay according to the present invention will screen or test a cell for one or more target proteins, e.g., two or more target proteins, such as three or more target proteins, four or more target proteins, five or more target proteins, etc.

A feature of the subject invention is that the target proteins are proteins that may be associated with cellular locomotion, e.g., of metastatic cells, and in certain embodiments are nucleus-associated ribbon-like structure proteins. By cellular locomotion protein is meant a protein that is involved or associated with cellular motility or movement from a first to a second location. In many embodiments, the cellular locomotion protein is a metastatic cellular locomotion protein, by which is meant that the protein is directly involved in the metastatic movement of a cell from one location to another, and therefore the metastatic phenotype of the cell.

In certain embodiments, the at least one target protein is a leading edge cellular locomotion protein. By leading edge cellular locomotion protein is meant that the protein, when present in a metastatic cell, localizes to vesicles near or proximal to the leading edge of the cell membrane. A representative leading edge cellular locomotion protein is Neurotrophic tyrosine kinase receptor type 2 (NTRK2/TrkB). Human NTRK2 is described in Nakagarawa et al., Genomics. 1995 Jan. 20; 25(2):538-46. The amino acid sequence and nucleic acid coding sequences therefore for human NTRK2 are deposited with Genbank at accession nos. U12140 and NM_{—}006180.

In certain embodiments, the at least one target protein is a nucleus-associated ribbon-like structure protein. Representative nucleus-associated ribbon-like structure proteins of interest include, but are not limited to: leukotriene B4 12-hydroxydehydrogenase (LTB4DH); Pregnancy-induced growth inhibitor (OKL38); Hs.516830 (C20orf139); and Tripartite-containing motif 29 (TRIM29). Human LTB4DH is described in Yokomizu, T et al. (1996) J Biol Chem 271:2844-2850. The amino acid sequence and nucleic acid coding sequence therefore for human LTB4DH is deposited with Genbank at accession no. NM_{—}012212. Human OKL38 is described in Huynh, H. et al. (2001) Endocrinology 142:3607-3615. The amino acid sequence and nucleic acid coding sequence therefore for human OKL38 is deposited with Genbank at accession no. NM_{—}013370. The amino acid sequence and nucleic acid coding sequence therefore for human Hs.516830 is deposited with Genbank at accession no. NM_{—}080725. Human TRIM29 is described in Kapp, L N et al. (1992) Am J Hum Genet. 51(1):45-54. The amino acid sequence and nucleic acid coding sequence therefore for human TRIM29 is deposited with Genbank at accession no. NM_{—}012101.

Also of interest as target proteins are proteins whose gene expression in known cancerous cells, e.g., lung cancer cells such as adenocarcinoma and squamous cell carcinoma cells, is similar to the gene expression of one of the above specific target cell proteins in such cells. For purposes of the invention, the expression of any two given genes is considered similar if, using the protocol described in Pearson correlation, the similarity of expression is at least about 0.70. Specific representative additional target proteins of interest include, but are not limited to: cyp4 proteins, including but not limited to: human cyp4F2 (described in Kikuta et al. (1999) DNA Cell Biol 18:723-730; and having an amino acid sequence and nucleic acid coding sequence therefore deposited with Genbank at accession no. NM_{—}001082); human cyp4F3 (described in Kikuta et al. (1993) J Biol Chem 268:9376-9380; and having an amino acid sequence and nucleic acid coding sequence therefore deposited with Genbank at accession no. NM_{—}000896); human cyp4F8 (described in Byland, J et al (1999) Biochem Biophys Res Comm 261(1):169-174; and having an amino acid sequence and nucleic acid coding sequence therefore deposited with Genbank at accession no. NM_{—}007253); human cyp4F11 (described in Cui, X et al. (2000) 68 (2):161-166; and having an amino acid sequence and nucleic acid coding sequence therefore deposited with Genbank at accession no. NM_{—}021187); and human cyp4F12 (described in Byland, J et al. (2001) Biochem Biophys Res Commun. 280(3):892-7; and having an amino acid sequence and nucleic acid coding sequence therefore deposited with Genbank at accession no. NM_{—}023944).

Also of interest as target proteins are homologs or proteins (or fragments thereof) of the above specific proteins, e.g., from the same or other species, i.e. other animals, where such homologs or proteins may be from a variety of different types of species, such as mammals, e.g., rodents, such as mice, rats; domestic animals, e.g., horse, cow, dog, cat; and humans. By homolog is meant a protein having at least about 35%, such as at least about 40% and including at least about 60% amino acid sequence identity to sequences of the specific proteins listed above, wherein in certain embodiments the homolog is a protein that has a sequence that is substantially the same as the sequence of one of the above specified target proteins, where two given sequences are considered to be substantially the same if they share a sequence similarity of at least about 75%, including at least about 80, 85, 90, 95, 97, 99% or higher, including 100% identify. Sequence similarity is calculated based on a reference sequence, which may be a subset of a larger sequence, such as a conserved motif, coding region, flanking region, etc. A reference sequence will usually be at least about 6 residues long, such as at least about 10 residues long, and may extend to the complete sequence that is being compared. Algorithms for sequence analysis are known in the art, such as BLAST, described in Altschul et al. (1990), J. Mol. Biol. 215:403-10, etc. Unless specified otherwise, all sequence identity values provided herein are determined using GCG (Genetics Computer Group, Wisconsin Package, Standard Settings, gap creation penalty 3.0, gap extension penalty 0.1).

In certain embodiments, a given cell or collection of cells is assayed for the presence of a subcellular structure made up of one or more (e.g., two or more) of the above described target proteins. The subcellular target structure of interest may be made up of two or more of the above target proteins, e.g., three or more of the above target proteins, including four or more of the above target proteins.

In certain representative embodiments, the cell is assayed for the presence of a nucleus-associated ribbon-like structure. The nucleus-associated ribbon-like structure for which a cell is screened in these embodiments is one that is made up of one or more of: leukotriene B4 12-hydroxydehydrogenase (LTB4DH); Pregnancy-induced growth inhibitor (OKL38); Hs.516830 (C20orf139); and Tripartite-containing motif 29 (TRIM29); where one or more additional proteins may also be present. This target nucleus piercing ribbon-like structure is further described in the Experimental section, below.

Cells can be assayed for presence of one or more target proteins according to a variety of different methods, the particular method employed not being critical to practice of the subject method. Representative methods of protein detection are now reviewed for convenience.

Individual target proteins, or composite structures thereof, can be determined in cells in biopsy material according to the subject methods by conventional methods well known to those of skill in the art. Such methods are described in many standard textbooks and laboratory manuals. For instance, the techniques for making and using antibody and other immunological reagents and for detecting particular proteins in samples using such reagents are described in CURRENT PROTOCOLS IN IMMUNOLOGY, Coligan et al., Eds., John Wiley & Sons, New York (1995). As another example, immunohistochemical methods for determining proteins in cells in tissues are described in Volume 2, Chapter 14 of CURRENT PROTOCOLS IN MOLECULAR BIOLOGY, Ausubel et al., Eds., John Wiley & Sons, Inc. (1994). Finally, Linnoila et al., A.J.C.P. 97(2): 235-243 (1992) and Peri et al., J. Clin. Invest. 92: 2099-2109 (1993). For instance, the amount of one or more of the above described target proteins in a sample can be determined in accordance with the invention by histochemical methods set out in Miyamoto et al., J. Urology 149: 1015-1019 (1993). As described therein, for instance, suitable biopsy material is obtained from a patient suspected of having benign or neoplastic tumor, e.g., benign lung hyperplasia or lung carcinoma, and immediately placed into 0.01M phosphate buffered saline. Thereafter, the material is immediately processed. It is mounted on a brass plate using rat liver homogenate as an adhesive. The material then is frozen in liquid nitrogen-cooled isopentane. Sections suitable for assay of target proteins in cells of the material are sectioned in a cryostat. Sections are obtained across the biopsy material, avoiding parts of the biopsy material that are damaged or deleteriously altered by the removal process.

Sections are dried at room temperature, fixed and then washed. Paraformaldehyde is a particularly useful fixative in this regard, but many other fixatives also can be used. The sections may be pretreated with hydrogen peroxide and a non-ionic detergent, such as Triton X-100. Also, sections may be incubated with a blocking solution to reduce non-specific binding. For instance, the sections may be incubated with goat blocking serum prior to incubation with a goat serum, goat antibody or goat antibody-derived reagent.

The target protein(s) then is visualized for determination in the samples using a target protein-specific binding reagent, such as a monoclonal or a polyclonal anti-target protein antibody. Binding of the target protein-specific reagent to cells in the sections may be determined directly, if the reagent has been conjugated to a detectable label, or using a second or additional reagents, such as a secondary antibody-enzyme conjugate.

In certain embodiments of the invention, the target protein-specific reagent is an antiserum, a polyclonal antibody, a derivative of a polyclonal antibody, a monoclonal antibody, a derivative of a monoclonal antibody or an engineered antibody, such as a single chain antibody. Derivatives of monoclonal and polyclonal antibodies include conjugates and fragments. Antibodies conjugated to detectable labels may be employed for these purposes. Among detectable labels are enzymes such as horseradish peroxidase. Among fragments employed in this regard are Fab fragments, F(ab′)₂ fragments and F(ab′) fragments.

Sections are incubated with target protein-specific reagent under conditions effective for the target protein-specific reagent to bind efficiently to target protein in the cells, while binding to other cellular components is inefficient; i.e., under conditions effective for the ratio of specific to non-specific binding to provide accurate determination of target protein content in cells of the biopsy material.

At the same time, control sections may be incubated under the same conditions with a corresponding reagent that is not specific for the target protein(s) to estimate background binding. For polyclonal immune serum, for instance, control sections can be incubated with pre-immune serum to monitor background, non-specific binding. After the incubation period, the specific reagent, and any reagent used in the controls, is removed, as by washing.

If the primary, target protein-specific reagent is detectably labeled, then the label may be determined and, thereby the target protein content of cells in the sample. In this case, controls preferably would be labeled and would be determined in like fashion. More often, and preferably, a secondary reagent is used to visualize binding of reagents on the sections, as described below.

After removing unbound specific and non-specific reagents, test and control sections are incubated with a secondary reagent that binds specifically to the primary, target protein specific reagent and its counterpart in the controls. For example, the secondary reagent is a biotinylated anti-antibody.

The sections are incubated with the secondary reagent under conditions for the reagent to bind efficiently to the primary reagent (and its counterpart in the controls) in the cells, while binding to other cellular components is inefficient; i.e., under conditions effective for the ratio of specific to non-specific binding to provide accurate determination of target protein content in cells of the biopsy material.

Thereafter, the unbound fraction of the secondary reagent is removed from the sections. The secondary reagent, and its counterpart in the controls, then is determined. If the secondary reagent comprises a detectable label, incubation with a tertiary reagent generally will not be necessary. However, use of a tertiary reagent comprising a detectable label is more commonly employed for immunocytochemical analysis, generally. Therefore, for illustrative purposes, the three-component assay is described here.

In certain embodiments, the relative staining of diseased and normal cells in a section is compared with staining in control cells. The control cells are reference standards which typify results obtained by a given procedure in normal cells, cells characteristic of benign tumors, and cells characteristic of malignant tumors (metastatic cells). Within any category, moreover, control cells may provide a graded or gradient series of characteristic standard or normal results. Target protein in control cells may be determined at the same time target protein is determined in cells of the biopsy sample, or at another time. In certain embodiments of the invention, target protein is determined in control cells which serve as a standard reference series for subsequent clinical assays.

As is known, suitable reagents and conditions for carrying out the determination of one or more target proteins in cells in biopsy samples are well known and readily available. A multiplicity of procedures and reagents can be effectively employed for this purpose. Such reagents and techniques are routinely employed by those of skill in the arts of immunocytochemistry, histopathology and cytology. Additional details regarding representative protocols that may be employed can be found in the Experimental section, below.

In certain embodiments, the methods may include a step in which the detected presence of at least one cellular locomotion protein is compared with a reference or control in order to characterize the metastatic propensity or state of the cell, as well as the tumor from which it was obtained/derived, e.g., to determine whether the tumor from which the cell was obtained is benign or malignant. The terms “reference” and “control” as used herein mean a standardized data set to be used to interpret the observed data of a given patient and assign a prognostic class thereto. The reference or control may be a data set that is obtained from a cell/tissue known to have the desired phenotype, e.g., metastatic phenotype, including specific stage of a disease, and therefore may be a positive reference or control profile. In addition, the reference/control may be from a cell/tissue known to not have the desired phenotype, and therefore be a negative reference/control.

In certain embodiments, the obtained data set is compared to a single reference/control to obtain information regarding the phenotype of the cell/tissue being assayed. In yet other embodiments, the obtained data set is compared to two or more different reference/controls to obtain more in depth information regarding the phenotype of the assayed cell/tissue. For example, the obtained data set may be compared to a positive and negative reference/control to obtain confirmed information regarding whether the cell/tissue has the phenotype of interest, e.g., a metastatic phenotype (e.g., characterized by the presence of a leading edge cellular locomotion protein and/or a nucleus associated ribbon-like structure).

The comparison of the obtained data set and the one or more reference/controls may be performed using any convenient methodology, where a variety of methodologies are known to those of skill in the art, e.g., by comparing digital images of the data sets, by comparing databases data, etc.

The comparison step results in information regarding how similar or dissimilar the obtained data set is to the control/references, which similarity/dissimilarity information is employed to determine the phenotype of the cell/tissue being assayed, e.g., whether the cell has a metastatic phenotype. For example, similarity with a positive control indicates that the assayed cell/tissue has a metastatic phenotype. Likewise, similarity with a negative control indicates that the assayed cell/tissue has a benign phenotype.

Depending on the type and nature of the reference/control(s) to which the obtained data set is compared, the above comparison step yields a variety of different types of information regarding the cell/tissue that is assayed. As such, the above comparison step can yield a positive/negative determination of a metastatic phenotype of an assayed cell/tissue.

In certain embodiments, the above-obtained information about the cell/tissue being assayed is employed to diagnose a host, subject or patient with respect to presence and/or stage of a neoplastic disease condition. As such, the above described methods find use in a variety of different applications, including the assessment or evaluation, e.g., in the form of a diagnosis, of the metastatic state or potential, e.g., propensity, of a cell and the tumor from which the cell is derived. As such, the subject methods find use in detecting the presence of a neoplastic disease state in a patient, as well in methods of staging such a disease state. For example, the subject methods may detect the presence of lung cancer in a patient, e.g., by determining that a cell obtained from a tumor found in the subject is metastatic because of the presence, and in certain embodiments location, of at least one target cellular locomotion protein, e.g., the presence of a nucleus-associated ribbon-like structure. The subject methods may also be employed in determining the stage of a particular lung cancer in a subject, and therefore be employed to make predictions about the outcome of the condition, e.g., make a lung cancer prognosis for the subject.

The subject methods may further find use in pharmacogenomic applications. In these applications, a subject/host/patient is first diagnosed for the presence and/or stage of a neoplastic disease condition using a protocol such as the diagnostic protocol described above. The subject is then treated using a pharmacological protocol, where the suitability of the protocol for a particular subject/patient is determined using the results of the diagnosis step. More specifically, where the identified phenotype is metastatic, an appropriate therapeutic treatment protocol is then employed to treat the patient is then determined and employed.

Screening Assays

Also provided by the subject invention are screening protocols and assays for identifying agents that modulate, e.g., inhibit or enhance, the production/activity of the above-described nucleus-associated ribbon-like structure. The screening methods include assays that provide for qualitative/quantitative measurements of the presence and/or location of the nucleus-associated ribbon-like structure. Assays of interest include assays that detect the presence or absence of the nucleus-associated ribbon-like structure in the presence and absence of a candidate inhibitor agent. The screening method may be an in vitro or in vivo format. In an exemplary screening protocol, cells are contacted with a candidate agent whose activity is being assayed, and the cells are then imaged, e.g., using the method described below in the experimental section, to detect the presence or absence of the nucleus-associated ribbon-like structure. The result is then compared to a control or reference, e.g., a like cell not exposed to the candidate agent, in order to obtain information about the activity of the assayed candidate agent.

A variety of different candidate agents may be screened by the above methods. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents identified in the above screening assays find use in a variety of applications, including the representative applications described below.

Also provided by the subject invention are methods for determining whether a subject suffering from a neoplastic condition will be responsive to a particular therapy. In embodiments of these methods, an expression profile is obtained from the subject suffering from the neoplastic condition and employed to determine whether the subject will be responsive to a particular therapy of interest, e.g., chromatin function inhibiting agent therapy.

In practicing these embodiments, a subject or patient sample, e.g., cells or collections thereof, e.g., tissues, is assayed to determine whether the host from which the assayed sample was obtained is responsive to a given therapy, e.g., chromatin function inhibiting agent therapy, as reviewed below. In practicing the subject diagnostic methods, the sample is assayed to obtain an expression profile for one or more genes, where the term expression profile is used broadly to include a genomic expression profile, e.g., an expression profile of mRNAs, or a proteomic expression profile, e.g., an expression profile of one or more different proteins.

In generating the expression profile, in many embodiments a sample is assayed to generate an expression profile that includes expression data for at least one gene/protein, usually a plurality of genes/proteins, where by plurality is meant at least two different genes/proteins, such as at least about 3, at least about 4, at least about 5 or more, including at least about 20 different genes/proteins or more, such as 50 or more, 100 or more, etc.

Genes/proteins of interest are genes/proteins that are differentially expressed or present at different levels in responders and non-responders for the particular disease condition and therapy therefor of interest. Genes of interest include the above described target genes (e.g., the five specific genes evaluated in the experimental section below), as well as the additional genes appearing in Table 3. In such embodiments, one can determine a cell mediated immunity profile for a subject, e.g., an expression pattern of genes associated with cell mediated immunity or some other measure of a subject's cell mediate immunity profile, and then compare this profile to a control (as defined below) to make a determination about the antineoplastic therapy responsiveness of the subject. In such embodiments, the method performed is a method of identifying or determining a profile or signature (or other parameter) for a subject, and then comparing this profile to a control to determine whether the subject will or will not be responsive to the antineoplastic therapy of interest.

In certain embodiments, at least one of the genes/proteins in the prepared expression profile is from one or more of Tables 1, 2 and/or 3, where the expression profile may include expression data for 2, 4, 4, 10, 15 or more of, including all of, the genes/proteins listed in one or more of Tables 1 to 3. The number of different genes/proteins whose expression and/or quantity data, i.e., presence or absence of expression, as well as expression/quantity level, that are included in the expression profile that is generated may vary, but is typically at least 2, and in many embodiments ranges from 2 to about 15 or more, sometimes from 3 to about 10 or more.

In certain embodiments, the expression profile obtained is a genomic or nucleic acid expression profile, where the amount or level of one or more nucleic acids in the sample is determined. In these embodiments, the sample that is assayed to generate the expression profile employed in the diagnostic methods is one that is a nucleic acid sample. The nucleic acid sample includes a plurality or population of distinct nucleic acids that includes the expression information of the phenotype determinative genes of interest of the cell or tissue being diagnosed. The nucleic acid may include RNA or DNA nucleic acids, e.g., mRNA, cRNA, cDNA etc., so long as the sample retains the expression information of the host cell or tissue from which it is obtained. The sample may be prepared in a number of different ways, as is known in the art, e.g., by mRNA isolation from a cell, where the isolated mRNA is used as is, amplified, employed to prepare cDNA, cRNA, etc., as is known in the differential expression art. The sample is typically prepared from a cell or tissue harvested from a subject to be diagnosed, e.g., via biopsy of tissue, using standard protocols, where cell types or tissues from which such nucleic acids may be generated include any tissue in which the expression pattern of the to be determined phenotype exists, including, but not limited to, disease cells or tissue, etc.

The expression profile may be generated from the initial nucleic acid sample using any convenient protocol. While a variety of different manners of generating expression profiles are known, such as those employed in the field of differential gene expression analysis, one representative and convenient type of protocol for generating expression profiles is array-based gene expression profile generation protocols. Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acid provides information regarding expression for each of the genes that have been probed, where the expression information is in terms of whether or not the gene is expressed and, typically, at what level, where the expression data, i.e., expression profile, may be both qualitative and quantitative.

Alternatively, non-array based methods for quantitating the levels of one or more nucleic acids in a sample may be employed, including quantitative PCR, and the like.

Where the expression profile is a protein expression profile, any convenient protein quantitation protocol may be employed, where the levels of one or more proteins in the assayed sample are determined. Representative methods include, but are not limited to: proteomic arrays, flow cytometry, standard immunoassays, etc.

Following obtainment of the expression profile from the sample being assayed, the expression profile is compared with a reference or control profile to make a diagnosis regarding the therapy responsive phenotype of the cell or tissue, and therefore host, from which the sample was obtained/derived. The terms “reference” and “control” as used herein mean a standardized pattern of gene expression or levels of expression of certain genes to be used to interpret the expression signature of a given patient and assign a prognostic class The reference or control profile may be a profile that is obtained from a cell/tissue known to have the desired phenotype, e.g., responsive phenotype, and therefore may be a positive reference or control profile. In addition, the reference/control profile may be from a cell/tissue known to not have the desired phenotype, and therefore be a negative reference/control profile.

In certain embodiments, the obtained expression profile is compared to a single reference/control profile to obtain information regarding the phenotype of the cell/tissue being assayed. In yet other embodiments, the obtained expression profile is compared to two or more different reference/control profiles to obtain more in depth information regarding the phenotype of the assayed cell/tissue. For example, the obtained expression profile may be compared to a positive and negative reference profile to obtain confirmed information regarding whether the cell/tissue has the phenotype of interest.

The comparison of the obtained expression profile and the one or more reference/control profiles may be performed using any convenient methodology, where a variety of methodologies are known to those of skill in the array art, e.g., by comparing digital images of the expression profiles, by comparing databases of expression data, etc. Patents describing ways of comparing expression profiles include, but are not limited to, U.S. Pat. Nos. 6,308,170 and 6,228,575, the disclosures of which are herein incorporated by reference. Methods of comparing expression profiles are also described above.

The comparison step results in information regarding how similar or dissimilar the obtained expression profile is to the control/reference profiles, which similarity/dissimilarity information is employed to determine the phenotype of the cell/tissue being assayed. For example, similarity with a positive control indicates that the assayed cell/tissue has a responsive phenotype. Likewise, similarity with a negative control indicates that the assayed cell/tissue has a non-responsive phenotype.

Depending on the type and nature of the reference/control profile(s) to which the obtained expression profile is compared, the above comparison step yields a variety of different types of information regarding the cell/tissue that is assayed. As such, the above comparison step can yield a positive/negative determination of a responsive phenotype of an assayed cell/tissue.

In many embodiments, the above-obtained information about the cell/tissue being assayed is employed to diagnose a host, subject or patient with respect to responsive to a particular therapy, such as chromatin function inhibiting agent therapy, as described above.

The subject methods further find use in pharmacogenomic applications. In these applications, a subject/host/patient is first diagnosed for the presence of absence of a responsive phenotype using a protocol such as the diagnostic protocol described in the preceding section.

The subject is then treated using a pharmacological protocol, where the suitability of the protocol for a particular subject/patient is determined using the results of the diagnosis step. More specifically, where the identified phenotype is responsive, an appropriate chromatin function inhibiting agent therapy treatment protocol is then employed to treat the patient. Alternatively, where a patient is identified as having a non-responsive phenotype, non-chromatin function inhibiting agent therapy protocols are then employed.

Therapeutic Methods

As summarized above and described in more detail below, the subject invention provides therapeutic methods. In a broad sense, such methods in certain embodiments may be viewed as methods of modulating movement of genomic material in a cell. In certain embodiments, methods may be viewed as methods that modulate the integrity of the genome. In certain embodiments, the methods may be viewed as methods of modulating cellular movement from a first to a second location in a host, and in particular cellular movement that the subject methods provide a means for changing, e.g., enhancing, inhibiting, impairing, altering, etc., genomic material movement, cellular locomotion, in a host, as compared to a control. In certain embodiments, the modulation is an inhibition, e.g., at least a reduction up to and including a complete cessation, of movement of the target cell in the subject. The desired modulation may result from one or more different mechanisms. For example, the subject invention may change/influence/alter cellular movement through modulation, either directly or indirectly, of the transcription or expression of genes encoding the target proteins identified above in cell. Alternatively, the modulation may be achieved through changing, e.g., inhibition, of the activity of one or more target cellular locomotion proteins in a cell. Accordingly, in certain embodiments an agent may be employed that acts by interfering or modulating the activity of one or more target proteins, e.g., by inhibiting or impairing the formation of a functional nucleus-associated ribbon-like structure.

A variety of different types of molecules may be used as an active agent in a given method. As such, active agents of interest include, but are not limited to: small or low molecular weight compounds, peptides, polypeptides and proteins (including intrabodies); nucleic acids, e.g., antisense molecules, and the like. Of interest in certain embodiments are small molecule compounds. Naturally occurring or synthetic small molecule compounds of interest include numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, nucleic acids, fatty acids, steroids, purines, pyrimidines, derivatives, structural analogs or combinations thereof.

As mentioned above, the subject invention provides methods of inhibiting cellular locomotion. In such methods, the target cell is contacted with an effective amount an active agent, where representative agents are described in detail above. The method of contact may vary depending on the location/environment of the target cell and/or the specific nature of the inhibitory agent. For example, where the target cell is present in culture, contact may be achieved by introducing the agent into the culture medium. Alternatively, where the target cell is present in an animal, i.e., in vivo, contact may be achieved through administration of the agent to the animal. By effective amount is meant the amount required to achieve the desired result, i.e., inhibition of metastatic cell motility, where such amounts may readily be determined empirically.

By “effective amount” is meant a dosage sufficient to produce the desired result, e.g., an inhibition of cellular movement, or an improvement in a disease condition or the symptoms associated therewith associated with or resulting from unwanted cellular movement. The agent may be administered to the host using any convenient means capable of producing the desired result. Thus, the agent can be incorporated into a variety of formulations for therapeutic administration. More particularly, the agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the agent can be achieved in various ways, including oral, buccal, rectal, parenteral, intraperitoneal, intradermal, transdermal, intracheal, etc., administration. In pharmaceutical dosage forms, the agent may be administered alone or in combination with other pharmaceutically active compounds. The following methods and excipients are merely exemplary and are in no way limiting.

For oral preparations, the agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agent can be utilized in aerosol formulation to be administered via inhalation. The agent can be formulated into pressurized acceptable propellants such as dichlorodifluoromethane, propane, nitrogen and the like.

Furthermore, the agent can be made into suppositories by mixing with a variety of bases such as emulsifying bases or water-soluble bases. The agents can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing active agent. Similarly, unit dosage forms for injection or intravenous administration may comprise the active agent in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

The subject methods of these embodiments find use in a variety of different applications, including the treatment of a variety of different disease conditions associated with the presence of metastatic cells, e.g., neoplastic disease conditions. As such, one disease condition of particular interest is neoplastic diseases, particularly those characterized by the presence of metastatic tumors. By treatment is meant at least an amelioration of the symptoms associated with the disease condition afflicting the host, where amelioration is used in a broad sense to refer to at least a reduction in the magnitude of a parameter, e.g. symptom, associated with the pathological condition being treated, such as size of tumor, rate of growth of tumor, spread of tumor, etc. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Accordingly, the methods of these embodiments of the present invention may be applied to the treatment of a variety of cancers, e.g., of epithelial cell origin. Among these are metastatic cancers of breast, lung, colon, bladder, lung, gastrointestinal track, endometrium, tracheal-bronchial tract, pancreas, liver, uterus, nasopharynges and the skin. In certain embodiments, the methods are employed in the treatment of lung cancer, including lung cancer of epithelial cell origin. Metastatic potential of lung cancers can be inhibited by methods of the invention. In certain embodiments, the methods of subject invention are employed to treat lung cancer, where specific lung cancers of interest include, but are not limited to: adenocarcinoma and squamous cell carcinoma.

The subject methods may be used in conjunction with other treatment modalities. Other common treatment modalities are discussed below specifically by reference to lung cancer. It will be appreciated that similar consideration will apply to treatment of other metastatic cancers. The present invention may be used in conjunction with any current or future therapy. Specific representative additional therapies of interest include surgery, radiation, hormonal therapy, chemotherapy, immunotherapy, cryotherapy, etc.

In certain embodiments, the subject methods include use of chromatin function inhibiting agents, including, but not limited to: microtubule inhibitors, e.g., agents that inhibit dividing cells by interfering with the mitotic spindle, such as taxoids, e.g. paclitaxel and functional analogs, e.g., synthetic derivatives, thereof. Of interest in certain embodiments is paclitaxel. Of interest in other embodiments is a paclitaxel analog, including but not limited to, the analogs disclosed in U.S. Pat. No. 6,800,660, the disclosure of which is herein incorporated by reference, which analogs are referred to collectively as taxanes.

In these embodiments, the agent that modulates the activity of at least one target protein associated with cellular locomotion is administered in combination with the chromatin function inhibiting agent (e.g., paclitaxel or analog thereof). By “in combination with” is meant that an amount of the first locomotion modulatory agent is administered together with an amount of the second chromatin function inhibiting agent. In certain embodiments, the first and second agents are administered sequentially, e.g., where the first agent is administered before or after the second agent. In yet other embodiments, the first and second agents are administered simultaneously, e.g., where the first and second agents are administered at the same time as two separate formulations or are combined into a single composition that is administered to the subject. Regardless of whether the first and second agents are administered sequentially or simultaneously, as illustrated above, the agents are considered to be administered together or in combination for purposes of the present invention. Routes of administration of the two agents may vary, where representative routes of administration are described in greater detail below.

Pharmaceutical Compositions

Also provided are pharmaceutical compositions containing the active agent(s), employed in the subject methods. The active agent(s), e.g., in the form of a pharmaceutically acceptable salt, can be formulated for oral or parenteral administration for use in the subject methods, as described above. In certain embodiments, e.g., where the compounds are administered as separate formulations (such as in those embodiments where they are administered sequentially), separate or distinct pharmaceutical compositions—each containing a different active agent, are provided. In yet other embodiments, a single formulation that includes all of the active agents (i.e., one composition that includes both active agents) is provided.

By way of illustration, the active compound(s) can be admixed with conventional pharmaceutical carriers and excipients (i.e., vehicles) and used in the form of aqueous solutions, tablets, capsules, elixirs, suspensions, syrups, wafers, and the like. Such pharmaceutical compositions contain, in certain embodiments, from about 0.1 to about 90% by weight of the active compound, and more generally from about 1 to about 30% by weight of the active compound. The pharmaceutical compositions may contain common carriers and excipients, such as corn starch or gelatin, lactose, dextrose, sucrose, microcrystalline cellulose, kaolin, mannitol, dicalcium phosphate, sodium chloride, and alginic acid. Disintegrators commonly used in the formulations of this invention include croscarmellose, microcrystalline cellulose, corn starch, sodium starch glycolate and alginic acid.

A liquid composition will generally consist of a suspension or solution of the compound or pharmaceutically acceptable salt in a suitable liquid carrier(s), for example, ethanol, glycerine, sorbitol, non-aqueous solvent such as polyethylene glycol, oils or water, with a suspending agent, preservative, surfactant, wetting agent, flavoring or coloring agent. Alternatively, a liquid formulation can be prepared from a reconstitutable powder.

For example, a powder containing active compound, suspending agent, sucrose and a sweetener can be reconstituted with water to form a suspension; and a syrup can be prepared from a powder containing active ingredient, sucrose and a sweetener.

A composition in the form of a tablet can be prepared using any suitable pharmaceutical carrier(s) routinely used for preparing solid compositions. Examples of such carriers include magnesium stearate, starch, lactose, sucrose, microcrystalline cellulose and binders, for example, polyvinylpyrrolidone. The tablet can also be provided with a color film coating, or color included as part of the carrier(s). In addition, active compound can be formulated in a controlled release dosage form as a tablet comprising a hydrophilic or hydrophobic matrix.

A composition in the form of a capsule can be prepared using routine encapsulation procedures, for example, by incorporation of active compound and excipients into a hard gelatin capsule. Alternatively, a semi-solid matrix of active compound and high molecular weight polyethylene glycol can be prepared and filled into a hard gelatin capsule; or a solution of active compound in polyethylene glycol or a suspension in edible oil, for example, liquid paraffin or fractionated coconut oil can be prepared and filled into a soft gelatin capsule.

Tablet binders that can be included are acacia, methylcellulose, sodium carboxymethylcellulose, poly-vinylpyrrolidone (Povidone), hydroxypropyl methylcellulose, sucrose, starch and ethylcellulose. Lubricants that can be used include magnesium stearate or other metallic stearates, stearic acid, silicone fluid, talc, waxes, oils and colloidal silica.

Flavoring agents such as peppermint, oil of wintergreen, cherry flavoring or the like can also be used. Additionally, it may be desirable to add a coloring agent to make the dosage form more attractive in appearance or to help identify the product.

The compounds of the invention and their pharmaceutically acceptable salts that are active when given parenterally can be formulated for intramuscular, intrathecal, or intravenous administration.

A typical composition for intramuscular or intrathecal administration will be of a suspension or solution of active ingredient in an oil, for example, arachis oil or sesame oil. A typical composition for intravenous or intrathecal administration will be a sterile isotonic aqueous solution containing, for example, active ingredient and dextrose or sodium chloride, or a mixture of dextrose and sodium chloride. Other examples are lactated Ringer's injection, lactated Ringer's plus dextrose injection, Normosol-M and dextrose, Isolyte E, acylated Ringer's injection, and the like. Optionally, a co-solvent, for example, polyethylene glycol, a chelating agent, for example, ethylenediamine tetraacetic acid, and an anti-oxidant, for example, sodium metabisulphite may be included in the formulation. Alternatively, the solution can be freeze dried and then reconstituted with a suitable solvent just prior to administration.

The compounds of the invention and their pharmaceutically acceptable salts which are active on rectal administration can be formulated as suppositories. A typical suppository formulation will generally consist of active ingredient with a binding and/or lubricating agent such as a gelatin or cocoa butter or other low melting vegetable or synthetic wax or fat.

The compounds of this invention and their pharmaceutically acceptable salts which are active on topical administration can be formulated as transdermal compositions or transdermal delivery devices (“patches”). Such compositions include, for example, a backing, active compound reservoir, a control membrane, liner and contact adhesive. Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference in its entirety. Such patches may be constructed for continuous, pulsatile, or on demand delivery of pharmaceutical agents.

Optionally, the pharmaceutical composition may contain other pharmaceutically acceptable components, such a buffers, surfactants, antioxidants, viscosity modifying agents, preservatives and the like. Each of these components is well-known in the art. See, for example, U.S. Pat. No. 5,985,310, the disclosure of which is herein incorporated by reference.

Other components suitable for use in the formulations of the present invention can be found in Remington's Pharmaceutical Sciences, Mace Publishing Company, Philadelphia, Pa., 17th ed. (1985).

Kits & Systems

Also provided are kits and systems that find use in practicing the subject methods, as described above. For example, kits and systems for practicing the subject methods may include one or more pharmaceutical formulations. As such, in certain embodiments the kits may include a single pharmaceutical composition, present as one or more unit dosages, where the composition may include one or more expression/activity inhibitor compounds. In yet other embodiments, the kits may include two or more separate pharmaceutical compositions, each containing a different active compound.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The term “system” as employed herein refers to a collection of two or more different active agents, present in a single or disparate composition, that are brought together for the purpose of practicing the subject methods.

The following examples are offered by way of illustration and not by way of limitation.

### Experimental

I. Materials and Methods

A. Immunofluorescence Microscopy.

HBEC were grown to 70% confluency on 1 well-chambered coverglass (Lab-Tek II, Nunc, #1.5). For all experiments except the localization of LTB4DH and Hs.516830 to the centriole in D51 cells, which required a stringent wash prior to fixation, standard fixation conditions were used. For standard conditions, culture media was removed and the cells were fixed directly in PBS containing 3% formaldehyde for 20 minutes. For stringent conditions, cells were rinsed quickly in PHEM buffer (60 mM Pipes, 25 mM Hepes, 10 mM EGTA, 1 mM Mg-acetate, pH 6.9), lysed for 2 minutes in PHEM containing 0.5% Triton X100, and fixed in PHEM containing 3% formaldehyde for 20 minutes. All steps subsequent to cell fixation were identical. Cells were incubated with PBS containing 50 mM NH₄Cl for 10 minutes, permeabilized in PBS containing 0.1% Triton X-100 at RT for 3 minutes, and blocked one hour in block buffer (PBS containing 0.75% BSA [GIBCO]). Primary antisera was diluted in block buffer to a final concentration of 1-10 ug/ml, depending on the antibody, and incubated with cells for 1.5 hours at room temperature. Conjugated secondary antibodies (diluted in block buffer to 2 ug/ml) were incubated for 45 minutes at room temperature. Phalloidin conjugated to rhodamine (Molecular Probes, 1 unit/ml final) was included during secondary antibody incubation where indicated in the figure legend. DNA was stained with 300 nM DAPI (Molecular Probes) in PBS for 5 minutes, and the cells were mounted in Prolong Antifade (Molecular Probes). Wide-field fluorescence microscopy (DeltaVision, Applied Precision) and subsequent deconvolution was used for all images that dealt with fixed cells. A 0.2-micron z-plane was used throughout. Three-dimensional rendering of stacked images was done on Volocity 2.0 software (Improvision).

B. Cell Cultures.

Primary cell cultures of human bronchial epithelial cells (HBEC) were purchased commercially (Cambrex, no retinoic acid) from two male donors and grown in BEGM medium (Cambrex) according to specifications. D51 cell line was derived from human adenocarcinoma of the lung (a kind gift from 1. Petersen, Charite, Berlin) and grown in RPMI 1640 containing 10% fetal bovine serum (Gibco). The human embryonic kidney cell line 293T was grown in DMEM/10% FBS (Gibco). MCF10A were grown in DMEM/F12 (Gibco) supplemented with L-glutamine, 2 ml bovine pituitary extract (Clonetics), 0.5 mg/L hydrocortisone (Clonetics), 24 mg/L insulin (Gibco), 0.5 ml gentamycin (Clonetics) and 100 ng/ml EGF (Peprotech). Whole cell protein extracts used for western blot analysis were obtained by resuspending adherent cells directly in SDS sample buffer containing 100 mM DTT. Unless otherwise indicted, cells were grown on cell culture inserts (25 mm, uncoated, Corning).

C. Microtubule-Interacting Drugs.

One hour prior to cell fixation, HBEC grown on coverglass or Transwell inserts (Corning) were washed twice in PBS and resuspended in BEGM containing 20 uM (5 ug/ml) nocodazole for 25 minutes at 37 C. Cells were quickly washed twice in PBS, resuspended in fresh BEGM without the drug for additional 25 minutes at 37 C, and immediately fixed as described above. HBEC or MCF10A was treated with 1 uM paclitaxel in cell culture media for 9 hours.

D. Antisera.

Polyclonal antisera were generated against two or three peptides from TRIM29 (MEAADASRSNGSSPEARDAR (SEQ ID NO:01), ELHLKPHLEGAAFRDHQ (SEQ ID NO:02), EGEGLGQSLGNFKDDLLN (SEQ ID NO:03)), Hs.516830 (RETIPAKLVQSTLSDLR (SEQ ID NO:04), DPAKVQSLVDTIREDPD (SEQ ID NO:05), LTB4DH (RVAAKRLKEGDTMMG (SEQ ID NO:06), KTVESLEETLKKASPDGYD (SEQ ID NO:07), TTHSISDGKDLEKLLTE (SEQ ID NO:08), and OKL38 (EYHKVHQMMREQSILSPSPYEGYR (SEQ ID NO:09), RHQLLCFKEDCQAVFQDLEGVEK (SEQ ID NO:10), DLEVKDWMQKKRRGLRNSR (SEQ ID NO:11)) (all Applied Genomics Inc., Huntsville, Ala.). All 4 polyclonal antisera were peptide affinity-purified. Secondary antibodies were obtained from Jackson ImmunoResearch. Monoclonal antiserum to alpha-tubulin was from Santa Cruz Biotechnology (sc-5286).

E. Retroviral Vectors and Infection.

PCR was used to construct a flexible linker (amino acids GGGGSGGGGS) attached to the carboxyl-terminal end of full-length TRIM29 cDNA (BC017352, nucleotides 64-1830) using the following primers: 5′-ggatccATGGAAGCTGCAGATGCCTCCAGG-3′ (SEQ ID NO:12) and 5′-accggtgtgcatcctccgccgccgcatcctccgccgccggcggcTGGGGCTTCGTTGGACCCAATCCCGTT-3′ (SEQ ID NO:013). PCR product was inserted into pEGFP-N1 (Promega) using the BamH1/Age1 restriction sites. The TRIM29-linker-EGFP DNA insert was cut out with BamHI/Not1 and inserted into the BamHI/Not1 sites within the retroviral vector pIB2 (a gift from M. Davis, Stanford). PIB2 is based on the pBMN retroviral vectors (G. Nolan, Stanford)

The Phoenix amphotropic retroviral packaging system (a gift from G. Nolan, Stanford) is a 293T cell line that constitutively expresses gag-pol. Phoenix cells were transfected with lipofectamine 2000 (Invitrogen) according to manufacturer specifications. Co-transfection with pIB2, which contains the TRIM29-GFP fusion, and pCI-VSVg results in the production of amphotrophic retrovirus with a VSV-g pseudotype. OPTI-MEM transfection medium (Invitrogen) was changed 6 hours post-transfection and the cells were resuspended in DMEM/10% FCS. Retroviral supernatant was harvested 36 hours post-transfection. Virus-producing cells were grown at 32° C. in DMEM/10% FBS.

HBEC or 293T used for infection were grown to 70% confluency on 1-well coverglass. Cells were resuspended in 1.5 ml undiluted viral supernatant containing 15 ug/ml polybrene (Sigma/Aldrich). EGTA (2 mM final concentration) was added to HBEC cultures to inhibit intercellular adhesion. Following a 6 hour infection, cells were resuspended in BEGM and grown for an additional 2-3 days prior to fluorescence microscopy.

F. Live Cell Imaging.

A Zeiss LSM 510 confocal microscope, equipped with 63× oil Apochromat objective, was used for all live cell imaging. Argon mercury laser was set at 25% power and 0.5% transmission. Time series images were stacked and 3D rendered using Volocity 2.0 software.

II. Results

The five genes, whose names and predicted functional domains are listed below and in Table 1, attracted our interest for two reasons. First, we had previously found that expression levels for TRIM29 and LTB4DH mRNA correlated with metastatic, stage IV adenocarcinoma of the lung. Second, TRIM29, OKL38, NTKR2 and C20orf139 mRNA expression correlated strongly with LTB4DH in a subset of lung adenocarcinomas and/or squamous cell carcinomas.

1. LTB4DH is an NADPH-dependent oxidoreductase with a zinc-containing aldehyde dehydrogenase domain. The enzyme is capable of metabolizing the leukotriene LTB4.

2. Hs.516830 (C20orf139, henceforth RLA1, for ribbon-like antigen 1) is a poorly characterized gene whose predicted product contains a ParB-like nuclease domain. In prokaryotes, the ParB domain is involved in segregating bacterial genomes.

3. TRIM29 (ATDC) is one of a large family of proteins that contains zinc binding RING and B-box motifs. Using a GFP fusion construct, TRIM29 associated with fibrous structures in the osteosarcoma cell line U2OS.

TRIM29/ATDC suppressed the increased sensitivity to ionizing radiation in cells derived from patients with ataxia-telangiectasia (A-T). These cells contain mutations in ATM, the gene directly responsible for A-T. Both TRIM29/ATDC and ATM map to 11q22-q23. ATM is thought to control cell cycle signaling pathways required for a cellular response to DNA damage and genomic instability. TRIM29/ATDC was postulated to function downstream of ATM in the cellular response to DNA damage and genomic instability.

4. OKL38 contains a domain characteristic of NADPH-dependent oxidoreductases and synthases; in this respect it resembles LTB4DH. It also contains a thioredoxin reductase domain. The substrates and products of its putative enzymatic activity are unknown. Gene products resulting from several OKL38 splice variants inhibit proliferation, block DNA replication, and induce apoptosis in breast, lung, and kidney cell cultures.

5. NTRK2/TrkB is a receptor tyrosine kinase widely expressed in humans, especially in nervous tissue. Although this protein and its functions have been extensively characterized in the nervous system, remarkably little is known of any function for NTRK2/TrkB outside of the nervous system.

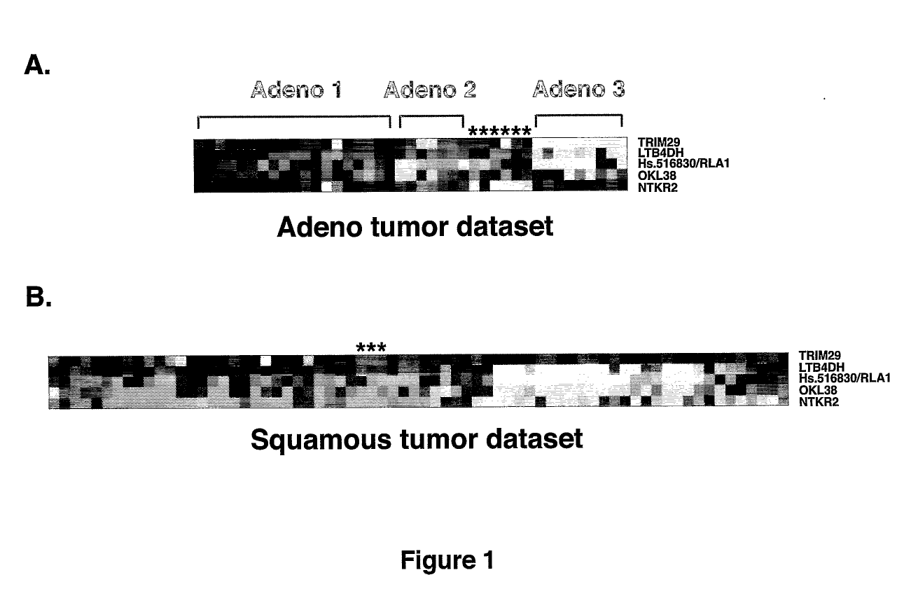

The expression patterns of these five genes across a set of 35 adenos of the lung and 67 squamous cell carcinomas of the lung and head/neck are shown graphically (FIG. 1). As can be seen from FIG. 1, Hs.516830, OKL38, and NTRK2/TrkB mRNA expression correlated strongly with expression of LTB4DH mRNA in a subset of squamous cell carcinomas (Table 2, see also FIG. 1 and Table 3 below). Hs.516830 mRNA expression also correlated strongly with LTB4DH mRNA in poor prognosis adeno of the lung (Table 2). TRIM29 mRNA was characteristically expressed in poor prognosis adeno and nearly all squamous cell tumors (FIG. 1). TRIM29 expression correlated strongly with expression of LTB4DH in the adeno dataset (Table 2). However, TRIM29 mRNA showed a very different gene expression pattern from LTB4DH mRNA in the squamous cell carcinoma dataset (data not shown). NTRK2/TrkB expression in adeno was relatively weak at the RNA level (FIG. 1, compare strong expression of NTRK2/TrkB in normal lung with relatively weak expression of NTRK2/TrkB in adeno group 3).

TABLE 1
Gene name, gene symbol, and predicted
functional domains for five proteins.

| GENE NAME (SYMBOL) | PREDICTED DOMAIN |
|---|---|
| Leukotriene B4 12-hydroxydehydrogenase | NADPH-dependent |
| (LTB4DH) | oxidoreductase |
| Pregnancy-induced growth inhibitor | NADPH-dependent |
| (OKL38) | oxidoreductase |
| Hs.516830 (C20orf139) | ParB-like nuclease domain |
| Tripartite-containing motif 29 (TRIM29) | B-Box-type zinc finger |
| Neurotrophic tyrosine kinase receptor | Tyrosine kinase, catalytic |
| type 2 (NTRK2/TrkB) | |

TABLE 2
Using pearson correlation, LTB4DH (clone W72246) showed
a similar gene expression pattern to Hs.516830, OKL38,
TRIM29, and NTRK2/TrkB across the adeno (left) and
squamous (right) tumor datasets. Numbers in parentheses
indicate the corrlelation to LTB4DH.

| | |
|---|---|
| ADENO TUMORS | SQUAMOUS TUMORS |

| | | | |
|---|---|---|---|
| LTB4DH | 1.00 | LTB4DH | 1.00 |
| Hs.516830 | 0.75 | Hs.516830 | 0.87 |
| TRIM29 | 0.66 | OKL38 | 0.77 |
| | | NTRK2/TrkB | 0.70 |

TABLE 3

| | | |
|---|---|---|
| 0.96 | IMAGE: 14 [symbol] | leukotriene B4 12-hydroxydehydrogenase ∥ Hs.114670 ∥ AA876375 |
| 0.95 | IMAGE: 16 [symbol] | ∥ ∥ AI000804 |
| 0.95 | IMAGE: 15 [symbol] | ∥ ∥ AA913303 |
| 0.92 | IMAGE: 74 [symbol] | leukotriene B4 12-hydroxydehydrogenase ∥ Hs.114670 ∥ AA420455 |
| 0.9 | IMAGE: 20 [symbol] | ∥ ∥ AI364951 |
| 0.89 | IMAGE: 21 [symbol] | ∥ ∥ AI393075 |
| 0.87 | IMAGE: 17 [symbol] | chromosome 20 open reading frame 139 ∥ Hs.135056 ∥ H49601 |
| 0.87 | IMAGE: 18 [symbol] | cytochrome P450, family 4, subfamily F, polypeptide 3 ∥ Hs.106242 ∥ AI249090 |
| 0.83 | IMAGE: 89 [symbol] | phosphogluconate dehydrogenase ∥ Hs.392837 ∥ AA598759 |
| 0.82 | IMAGE: 16 [symbol] | cytochrome P450, family 4, subfamily F, polypeptide 11 ∥ Hs.187393 ∥ AA991369 |
| 0.82 | IMAGE: 14 [symbol] | epidermal growth factor receptor pathway substrate 8 ∥ Hs.2132 ∥ H13622 |
| 0.82 | IMAGE: 12 [symbol] | cytochrome P450, family 4, subfamily F, polypeptide 12 ∥ Hs.180570 ∥ R11209 |
| 0.81 | IMAGE: 15 [symbol] | Homo sapiens, clone IMAGE: 5311297, mRNA ∥ Hs.116524 ∥ AA931721 |
| 0.81 | IMAGE: 15 [symbol] | ESTs ∥ Hs.128803 ∥ AA975760 |
| 0.81 | IMAGE: 78 [symbol] | thioredoxin reductase 1 ∥ Hs.13046 ∥ AA453335 |
| 0.81 | IMAGE: 12 [symbol] | cytochrome P450, family 4, subfamily F, polypeptide 12 ∥ Hs.180570 ∥ T98002 |
| 0.8 | IMAGE: 28 [symbol] | solute carrier family 7, (cationic amino acid transporter, y+ system) member |
| | | 11 ∥ Hs.6682 ∥ N59336 |
| 0.8 | IMAGE: 21 [symbol] | cytochrome P450, family 4, subfamily F, polypeptide 8 ∥ Hs.268554 ∥ AI401776 |
| 0.79 | IMAGE: 48 [symbol] | carboxylesterase 1 (monocyte/macrophage serine esterase 1) ∥ Hs.76688 ∥ |
| | | AA043416 |
| 0.79 | IMAGE: 14 [symbol] | ESTs ∥ Hs.128803 ∥ AA865075 |
| 0.79 | IMAGE: 29 [symbol] | aldo-keto reductase family 1, member C1 (dihydrodiol dehydrogenase 1; 20-alpha |
| | | (3-alpha)-hydroxysteroid dehydrogenase) ∥ Hs.4311751 ∥ N74260 |
| 0.79 | IMAGE: 68 [symbol] | ESTs, Weakly similar to hypothetical protein FLJ20378 [Homo sapiens] |
| | | [H. sapiens] ∥ Hs.439145 ∥ AA235343 |
| 0.79 | IMAGE: 30 [symbol] | transmembrane 4 superfamily member 2 ∥ Hs.82749 ∥ N93505 |
| 0.78 | IMAGE: 73 [symbol] | hypothetical protein BC000282 ∥ Hs.8116 ∥ AA416631 |
| 0.78 | IMAGE: 58 [symbol] | ESTs, Weakly similar to hypothetical protein FLJ20489 [Homo sapiens] |
| | | [H. sapiens] ∥ Hs.114959 ∥ AA130861 |
| 0.78 | IMAGE: 50 [symbol] | microtubule-associated protein 1B ∥ Hs.103042 ∥ H17493 |
| 0.78 | IMAGE: 15 [symbol] | transaldolase 1 ∥ Hs.77290 ∥ AA955007 |
| 0.78 | IMAGE: 19 [symbol] | aldo-keto reductase family 1, member C1 (dihydrodiol dehydrogenase 1; 20-alpha |
| | | (3-alpha)-hydroxysteroid dehydrogenase) ∥ Hs.431175 ∥ R93124 |
| 0.78 | IMAGE: 44 [symbol] | glutathione reductase ∥ Hs.193974 ∥ AA777763 |
| 0.78 | IMAGE: 48 [symbol] | CDK5 regulatory subunit associated protein 2 ∥ Hs.32360 ∥ AA046569 |
| 0.77 | IMAGE: 14 [symbol] | aldo-keto reductase family 1, member C3 (3-alpha hydroxysteroid dehydrogenase, |
| | | type II) ∥ Hs.78183 ∥ AA916325 |
| 0.77 | IMAGE: 26 [symbol] | v-maf musculoaponeurotic fibrosarcoma oncogene homolog G (avian) ∥ |
| | | Hs.252229 ∥ N21609 |
| 0.77 | IMAGE: 72 [symbol] | pregnancy-induced growth inhibitor ∥ Hs.31773 ∥ AA292891 |
| 0.77 | IMAGE: 21 [symbol] | ESTs ∥ Hs.283884 ∥ H72425 |
| 0.77 | IMAGE: 36 [symbol] | glutamate-cysteine ligase, modifier subunit ∥ Hs.89709 ∥ W96179 |
| 0.77 | IMAGE: 27 [symbol] | malic enzyme 1, NADP(+)-dependent, cytosolic ∥ Hs.14732 ∥ N35825 |
| 0.77 | IMAGE: 18 [symbol] | aldo-keto reductase family 1, member B10 (aldose reductase) ∥ Hs.116724 ∥ |
| | | AI301329 |
| 0.77 | IMAGE: 82 [symbol] | carboxylesterase 1 (monocyte/macrophage serine esterase 1) ∥ Hs.76688 ∥ T68878 |
| 0.76 | IMAGE: 29 [symbol] | WD repeat endosomal protein ∥ Hs.109778 ∥ N64681 |
| 0.76 | IMAGE: 34 [symbol] | EGF-like-domain, multiple 5 ∥ Hs.5599 ∥ W67981 |
| 0.75 | IMAGE: 20 [symbol] | ESTs ∥ Hs.295131 ∥ R98790 |
| 0.75 | IMAGE: 24 [symbol] | aldo-keto reductase family 1, member C2 (dihydrodiol dehydrogenase 2; bile acid |
| | | binding protein; 3-alpha hydroxysteroid dehydrogenase, type III) ∥ |
| | | Hs.201967 ∥ AI924357 |
| 0.75 | IMAGE: 85 [symbol] | malic enzyme 1, NADP(+)-dependent, cytosolic ∥ Hs.14732 ∥ AA669689 |
| 0.75 | IMAGE: 47 [symbol] | a disintegrin and metalloproteinase domain 23 ∥ Hs.7164 ∥ H11006 |
| 0.74 | IMAGE: 78 [symbol] | tachykinin, precursor 1 (substance K, substance P, neurokinin 1, neurokinin 2, |
| | | neuromedin L, neurokinin alpha, neuropeptide K, neuropeptide gamma) ∥ |
| | | Hs.2563 ∥ AA446 [symbol] |
| 0.74 | IMAGE: 78 [symbol] | ATP-blnding cassette, sub-family C (CFTR/MRP), member 3 ∥ Hs.90786 ∥ AA429895 |
| 0.74 | IMAGE: 19 [symbol] | **aldo-keto reductase family 1, member C1 (dihydrodiol dehydrogenase 1; 20-alpha |
| | | (3-alpha)-hydroxysteroid dehydrogenase) ∥ Hs.431175 ∥ R96609 |
| 0.74 | IMAGE: 30 [symbol] | ESTs ∥ Hs.432317 ∥ N90403 |
| 0.74 | IMAGE: 71 [symbol] | carbonyl reductase 1 ∥ Hs.88778 ∥ AA280846 |
| 0.74 | IMAGE: 28 [symbol] | glutamate-cysteine ligase, catalytic subunit ∥ Hs.151393 ∥ N45129 |
| 0.74 | IMAGE: 53 [symbol] | CD83 antigen (activated B lymphocytes, immunoglobulin superfamily) ∥ Hs.79197 ∥ AA083671 |
| 0.73 | IMAGE: 85 [symbol] | cellular repressor of E1A-stimulated genes ∥ Hs.5710 ∥ T71991 |
| 0.73 | IMAGE: 15 [symbol] | ESTs ∥ Hs.128803 ∥ AA977453 |
| 0.73 | IMAGE: 74 [symbol] | calcium-binding tyroslne-(Y)-phosphorylation regulated (fibrousheathin 2) ∥ |
| | | Hs.314452 ∥ AA625956 |
| 0.73 | IMAGE: 50 [symbol] | hypothetical protein MGC13090 ∥ Hs.333389 ∥ AA150896 |
| 0.72 | IMAGE: 15 [symbol] | SAR1 protein ∥ Hs.110796 ∥ AA977449 |
| 0.72 | IMAGE: 19 [symbol] | carbonyl reductase 3 ∥ Hs.154510 ∥ AI352345 |
| 0.72 | IMAGE: 84 [symbol] | Homo sapiens, clone IMAGE: 5300264, mRNA ∥ Hs.284235 ∥ AA486499 |
| 0.72 | IMAGE: 25 [symbol] | peroxiredoxin 1 ∥ Hs.180909 ∥ AW071125 |
| 0.72 | IMAGE: 58 [symbol] | glutathione peroxidase 2 (gastrointestinal) ∥ Hs.2704 ∥ AA135152 |
| 0.72 | IMAGE: 74 [symbol] | glutathione reductase ∥ Hs.193974 ∥ AA417617 |
| 0.72 | IMAGE: 66 [symbol] | **glutathione S-transferase M2 (muscle) ∥ Hs.279837 ∥ AA232327 |
| 0.71 | IMAGE: 21 [symbol] | Homo sapiens cDNA FLJ37920 fis, clone CTONG1000181. ∥ Hs.213397 ∥ H69608 |
| 0.71 | IMAGE: 33 [symbol] | ESTs ∥ Hs.445074 ∥ R18805 |
| 0.7 | IMAGE: 14 [symbol] | membrane-spanning 4-domains, subfamily A, member 8B ∥ Hs.26638 ∥ AA829284 |
| 0.7 | IMAGE: 19 [symbol] | prostaglandin I2 (prostacyclin) synthase ∥ Hs.302085 ∥ AI304790 |
| 0.7 | IMAGE: 20 [symbol] | **ESTs, Highly similar to GSH1_HUMAN Glutamate--cysteine ligase catalytic subunit |
| | | (Gamma-glutamylcysteine synthetase) (Gamma-ECS) (GCS heavy chain) [H. sapiens |
| 0.7 | IMAGE: 84 [symbol] | glutathione S-transferase M4 ∥ Hs.348387 ∥ AA486570 |
| 0.7 | IMAGE: 24 [symbol] | alcohol dehydrogenase 7 (class IV), mu or sigma polypeptide ∥ Hs.389 ∥ AI863845 |
| 0.7 | IMAGE: 71 [symbol] | glutathione S-transferase M1 ∥ Hs.301961 ∥ AA290737 |
| 0.7 | IMAGE: 20 [symbol] | pre-B-cell colony-enhancing factor ∥ Hs.239138 ∥ AI335002 |
| 0.7 | IMAGE: 28 [symbol] | neurotrophic tyrosine kinase, receptor, type 2 ∥ Hs.47860 ∥ N63949 |
| 0.69 | IMAGE: 76 [symbol] | aldehyde dehydrogenase 3 family, member A2 ∥ Hs.159608 ∥ AA418697 |
| 0.69 | IMAGE: 89 [symbol] | ESTs, Highly similar to slit homolog 2 (Drosophila); slit (Drosophila) homolog 2 |
| | | [Homo sapiens] [H. sapians] ∥ Hs.432333 ∥ AA489463 |
| 0.69 | IMAGE: 29 [symbol] | GLI-Kruppel family member GLI2 ∥ Hs.111867 ∥ AI822076 |

Accordingly, a functional characterization of the following five gene products was performed: LTB4DH, TRIM29, OKL38, Hs.516830, and NTRK2/TrkB.

A. Antisera Specific for Each of Five Tumor Markers

Affinity-purified polyclonal antisera to select peptides were generated for four of five tumor markers, i.e., all of the markers but NTRK2/TrkB. Antiserum against NTRK2/TrkB protein was purchased commercially. Two cell cultures were used for the western blot analysis. Human bronchial epithelial cells (HBEC) are a primary cell culture obtained commercially. D51 is a cell line derived from patient 80-96 with poor prognosis lung adeno.

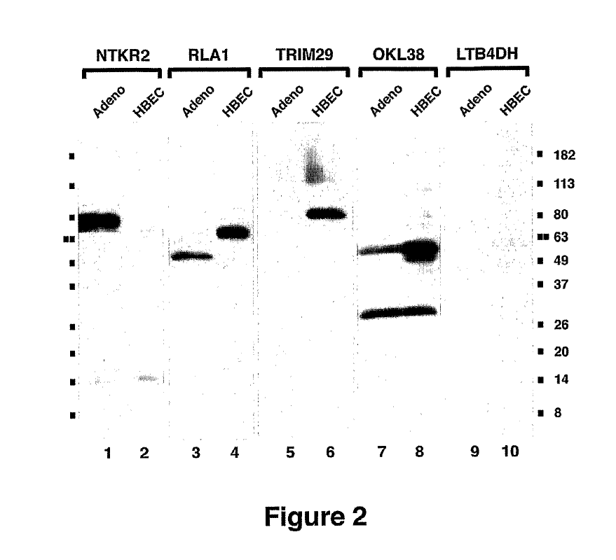

Three lines of evidence indicated that the antibodies were specific for the intended protein. First, western blot analysis revealed bands reacting with antisera against 4 of 5 of the given proteins, in both HBEC and D51 cell cultures. NTRK2/TrkB antiserum identified a protein band that migrated on SDS PAGE at 80 kD (FIG. 2, lanes 1,2); predicted molecular weight is ˜90 kD. Antibodies directed against Hs.516830 stained two prominent bands at ˜64 kD and ˜54 kD (FIG. 2, lanes 3,4). The predicted open reading frame for Hs.516830 is only 137 amino acids. The discrepancy in molecular weight for Hs.516830 may be due to a poorly characterized open reading frame for this hypothetical gene. Antibodies against TRIM29 identified a single band at ˜74 kD for HBEC (FIG. 2, lanes 5,6). Expression in D51 was very weak. TRIM29 protein has a predicted MW of 66 kD. OKL38 antiserum detected two bands at ˜57 kD and ˜30 kD on SDS-PAGE (FIG. 2, lanes 7, 8), in agreement with a previous, independent study. The LTB4DH polyclonal antibody gave poor results on both Western (FIG. 2, lanes 9,10) and paraffin-embedded tissue (data not shown).

The second line of evidence was obtained from the specificity of the antibodies in paraffin-embedded lung tumors. Immunohistochemical staining for TRIM29 and Hs.516830 proteins was observed preferentially in tumor cells for both lung adenocarcinoma and squamous cell carcinoma. The NTRK2/TrkB antibody specifically recognized the protein in squamous cell carcinoma of the lung by immunohistochemistry. Expression for these three proteins by immunohistochemistry followed closely the tissue distribution observed at the RNA level by DNA microarray analysis.

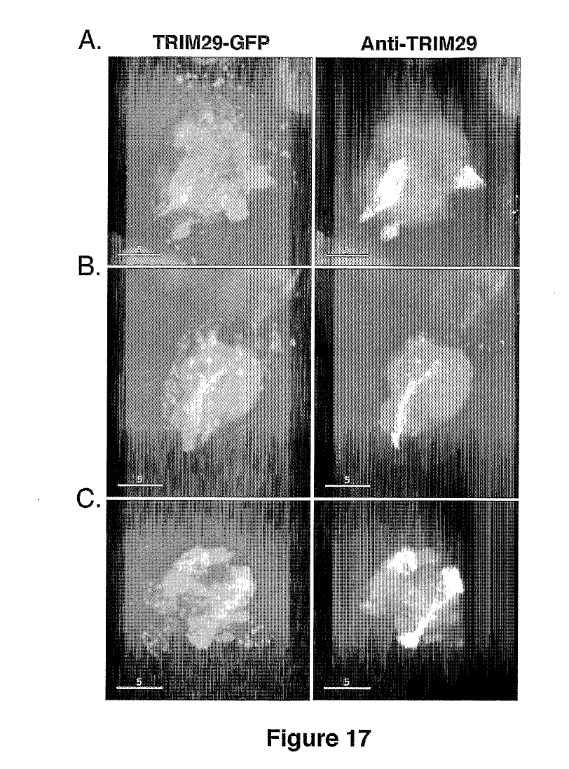

Finally, the subcellular localization of a TRIM29-GFP fusion protein closely matched the localization pattern detected by immunofluorescence staining using the TRIM29 antiserum (see FIG. 17 below).

B. Four of the Five Proteins Localized to a Novel Ribbon-Like Structure

To investigate mechanistically a possible role for the proteins in lung cancer, we initiated the characterization of all 5 proteins by using immunofluorescence microscopy in order to define their subcellular localization in cultured cells.

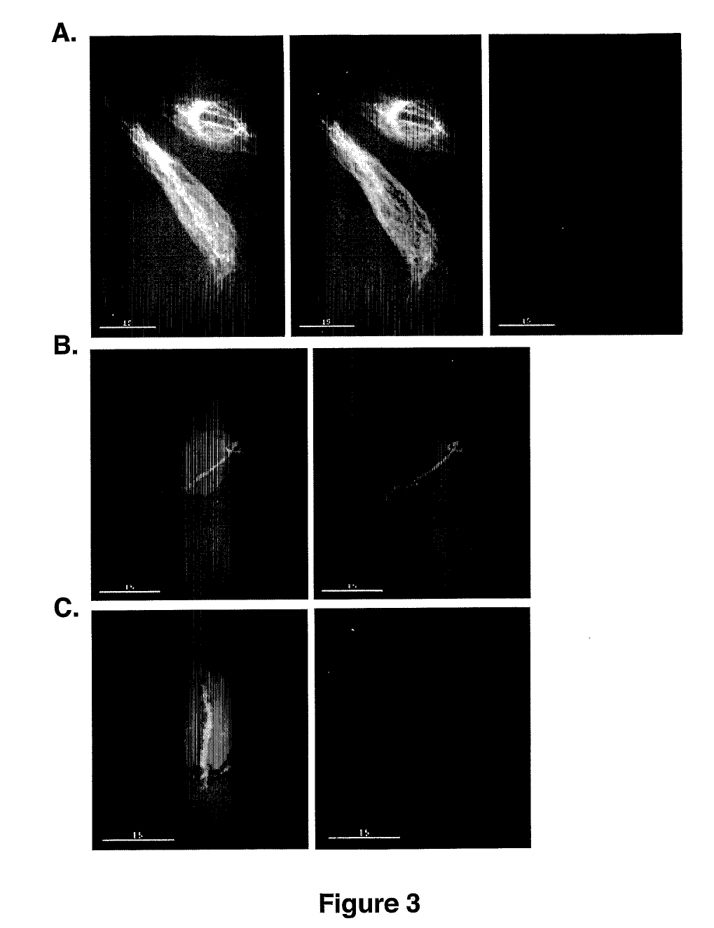

LTB4DH, Hs.516830, and TRIM29 localized to a ribbon-like structure in primary HBEC using immunofluorescence microscopy. LTB4DH antiserum identified a ribbon-like structure very close to the nucleus (FIG. 3A, green). Using an antibody to alpha-tubulin, microtubules did not co-localize with LTB4DH to the ribbon-like structure (FIG. 3A, red), despite the fact that we tried three different monoclonal antibodies to alpha-tubulin, including acetylated alpha-tubulin (data not shown). The ribbon-like structure containing LTB4DH spanned the length of the nucleus and ended very close to the nuclear periphery (FIG. 3B, green). In addition, the nucleus contained a groove or channel, suggesting the possibility that the ribbon-like structure pushed into the nucleus (FIG. 3C, green). Localization was confined to a minority of cells on the periphery of the culture.

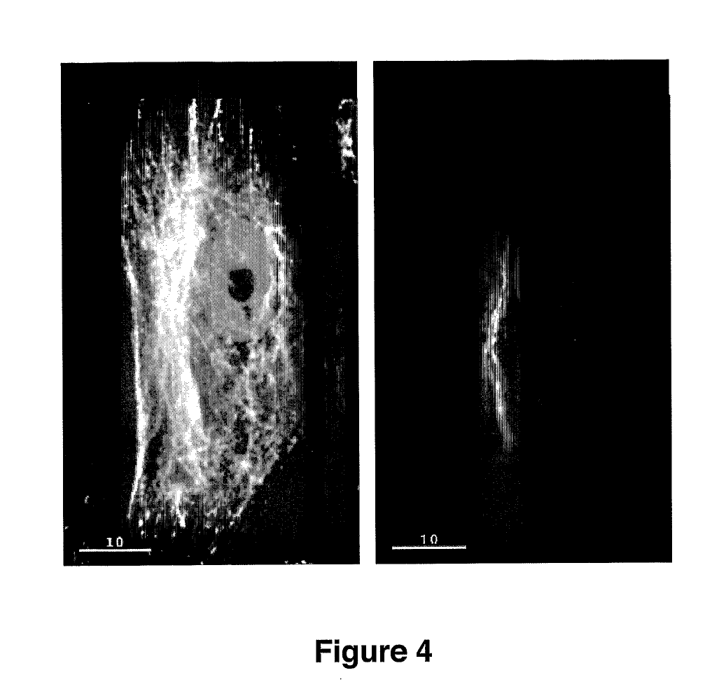

Like LTB4DH, Hs.516830 localized in primary HBEC cultures to a ribbon-like structure that originated in the vicinity of the nucleus. Like LTB4DH, localization of Hs.516830 to the ribbon-like structure was confined to a minority of cells on the periphery of the culture. Localization was observed across four stacked images (0.8 microns) within the upper part of the cell (FIG. 4A, left). Four stacked images (0.8 microns) across the lower part of the cell showed no evidence of the ribbon-like structure (FIG. 4A, right). There was also no evidence for co-localization of microtubules within the ribbon-like structure using double-labeling experiments (FIG. 4A, red). Unlike LTB4DH localization, Hs.516830 extended beyond the periphery of the nucleus. Viewed from the top, the nucleus (blue) contained a channel that ran parallel with the long axis of the cell (FIG. 4A, left). The data are consistent with the localization of Hs.516830, like LTB4DH, to a ribbon-like structure.

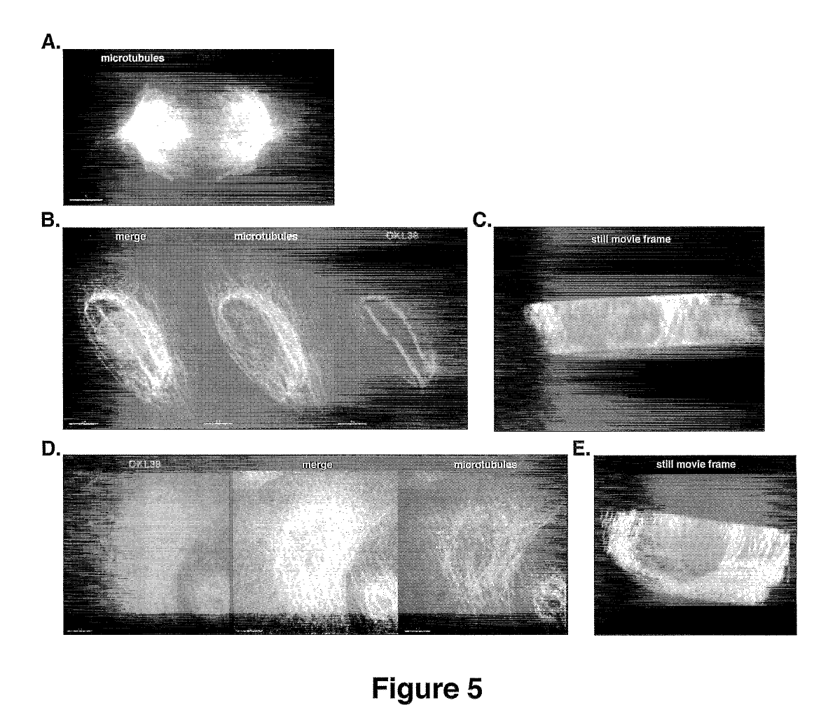

Using immunofluorescence microscopy, OKL38 localized most notably to microtubules in HBEC. OKL38 (green) co-localized with microtubules (red) to the mitotic spindle in HBEC (FIG. 5A). OKL38 expression was identical to microtubule fibers throughout the cell with one notable exception. In a small percentage of cells on the periphery of the culture, OKL38 localized in primary HBEC cultures to a ribbon-like structure (FIG. 5B, green) where microtubules were not observed (compare MT [red] with OKL38 [green]). The ribbon-like structure spanned well beyond the nuclear boundaries and intersected with microtubules close to the cell surface. Deconvolved images from the top to the bottom of the nucleus were stacked, 3D rendered, and displayed in movie format. The rendered three-dimensional object showed a ribbon-like structure on the top and bottom of the nucleus. A channel was clearly visible in the nucleus that corresponded to the placement of the ribbon structure. The virtual image was sliced perpendicular to the structure and the optical slice was rotated toward the viewer. OKL38 localized to a ribbon (green) that ran through the middle of the nucleus (blue) (FIG. 5C). Elimination of the green wavelength, representing the ribbon-like structure, revealed a channel within the nuclear compartment. Like LTB4DH and Hs.516830, localization of OKL38 to the ribbon-like structure was confined to a minority of cells on the periphery of the culture. Unlike LTB4DH, Hs.516830, and TRIM29, no evidence was found to suggest that OKL38 localized to the centriole.

The ribbon-like structures were mostly linear and parallel to the long axis of the cell. Surprisingly, we observed OKL38 localization to a ribbon-like structure that bent (FIG. 5E, green). Double-labeling experiments showed that OKL38 (green) co-localized with microtubules (red) throughout the cell except within a tripod structure in the center of the cell that intersected the nucleus (FIG. 5D). Deconvolved images from the top to the bottom of the cell were 3D rendered and displayed in movie format. The virtual image showed that OKL38 localized to a structure that nearly spanned from the top to the bottom of the nucleus (FIG. 5E). This novel structure is therefore more consistent with a ribbon than a string. These data indicated that OKL38, like LTB4DH and Hs.516830, localized to a ribbon-like structure and that the structure, unlike microtubules, actually pushed into the nucleus. We envision at least two possible mechanisms of entry into the nucleus. First, the novel structure impales the nuclear membrane and extends the length of the nucleus. Second, the structure extends the length of the nucleus and subsequently invaginates into the nuclear membrane.

C. Ribbon-Like Structure is Observed in Cells Differentially Sensitive to Nocodazole

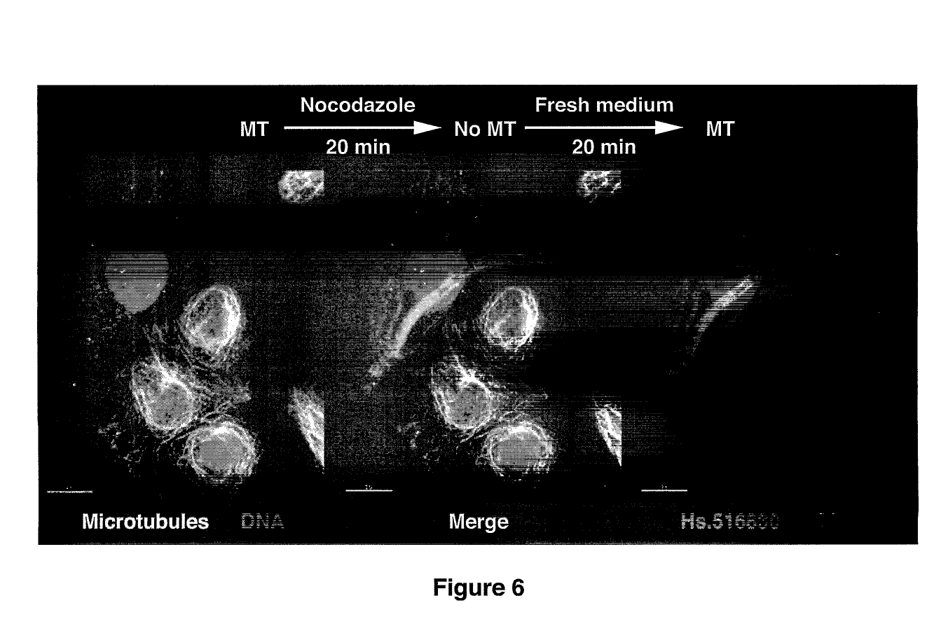

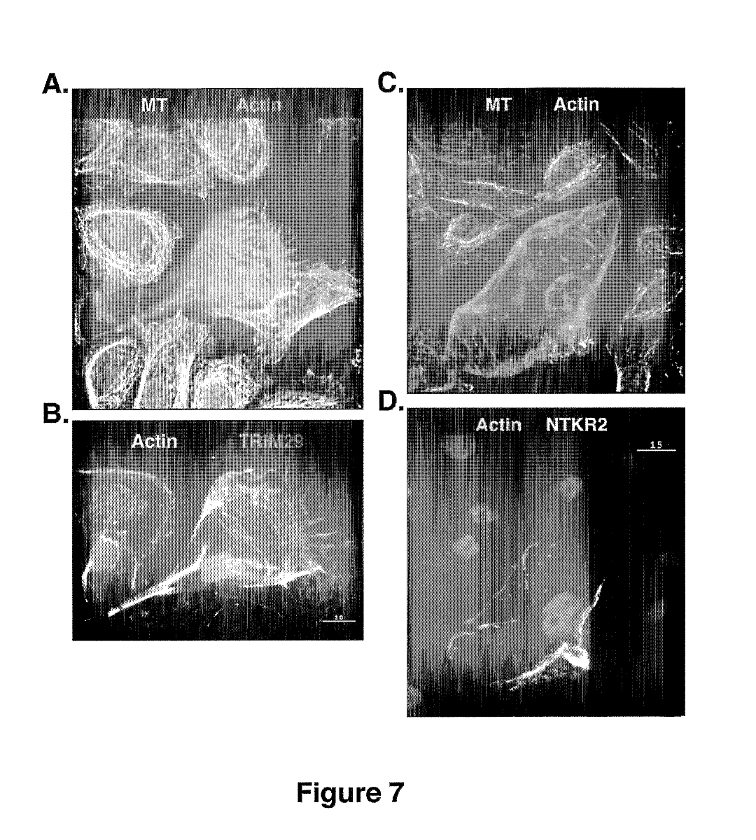

The MT destabilizing drug nocodazole was used to test whether microtubules were necessary for the structural integrity of the ribbon-like structure. HBEC were exposed to nocodazole for 20 minutes. Immunofluorescence microscopy using antisera to alpha tubulin showed that a 20 minute exposure to the drug was sufficient to eliminate cytoplasmic MT (data not shown). In addition, the ribbon-like structure was uncharacteristically absent, suggesting that the structure requires MT for stability. Cells were allowed to recover in fresh medium without nocodazole for another 20 minutes. We observed that the vast majority of cells reassembled microtubules during this recovery period. In several but not all cells on the periphery of the culture microtubules had not reassembled after 20 minutes recovery from nocodazole treatment (FIG. 7, left; observe nucleus without peripheral red MT fibers). Although we cannot exclude the possibility that these cells were undergoing apoptosis, the lack of chromatin fragmentation suggested that they were not. The cells that failed to re-assemble microtubules following nocodazole treatment preferentially expressed the ribbon-like structure, as indicated by localization with Hs.516830 antiserum (FIG. 6, right; observe cell on top). During the 20 minute recovery from the drug, the nascent ribbon-like structures were rather unrefined and sporatic. None of the cells in which MT had reassembled showed detectable Hs.516830 immunofluorescence that emanated from a ribbon-like structure (FIG. 6, center). Conditions that favor the structure and inhibit MT polymerization remain to be defined. These data indicated that the ribbon-like structure polymerized with kinetics similar to MT. In addition, select cells on the periphery of the cell culture, where we observed the structure, were different at least with respect to nocodazole sensitivity.

Using immunofluorescence microscopy and TRIM29 antiserum, we were unable to show localization for TRIM29 to any obvious steady-state structure in primary HBEC other than to the centriole. In particular, there was an absence of any ribbon-like structure that formed in cells on the periphery of the culture. Rather, we asked whether TRIM29 might be involved in the early stages of forming the ribbon-like structure. Nocodazole provided an opportunity to shrink and re-grow the ribbon-like structure in primary HBEC. Under these conditions, the role of TRIM29, if any, in the growth of the nascent structure could be investigated. Very similar to Hs.516830, there were several cells on the periphery of the culture that failed to re-grow microtubules (red) following nocodazole treatment (FIG. 7A, observe cell in center without peripheral red MT fibers). Actin fibers, on the other hand, were unaffected by nocodazole and present in all cells, irrespective of their position in the culture (FIG. 7A, compare MT [red] with actin [green]). Triple-labeling experiments localized simultaneously three proteins, actin (rhodamine), MT (FITC), TRIM29 (Cy5), and DNA (DAPI) within the same cell. Given RGB constraints, green and red colors were re-assigned in FIG. 7A and FIG. 7B. Results showed that the cell that failed to re-grow MTs (red) expressed TRIM29 (green) following nocodazole treatment (FIG. 7B). TRIM29 localized strongly to a cylindrical structure approximately 1.5 microns in length immediately outside the nucleus (FIG. 7B, green). In addition, TRIM29 also localized to a ribbon-like structure that emanated from the cylinder and reached toward the nucleus. In this cell, the actin cytoskeleton showed a lamellipodial protrusion, implying that the cell was possibly migrating/invasive (FIG. 7B, red). The ribbon-like structure pointed in the direction of the leading edge extension. Two observations are consistent with these data. First, TRIM29, like LTB4DH, OKL38, and Hs.516830, localized to a ribbon-like structure. Second, cells on the periphery of the culture expressing the ribbon-like structure were differentially sensitive to nocodazole.

Triple-labeling experiments localized simultaneously three proteins, actin (rhodamine), MT (FITC), NTRK2/TrkB (Cy5), and DNA (DAPI) within the same cell. Given RGB constraints, green and red colors were re-assigned in FIGS. 7A and 7B. Similar to TRIM29, NTRK2/TrkB localization in HBEC was most easily observed during short-term recovery from nocodazole. We found that select cells on the periphery of the culture failed to re-grow microtubules (red) following nocodazole treatment (FIG. 7C, observe cell in center without red fibers). Actin fibers, on the other hand, were unaffected by nocodazole and present in all cells (compare MT [red] with actin [green]). NTRK2/TrkB localization was observed across seven stacked images (1.4 microns) within the upper part of the cell (FIG. 7D, green, center cell with no M7). Actin filaments stained strongly on one side of the cell (red), consistent with actin protrusion of the cell edge. NTRK2/TrkB stained vesicular structures, consistent with localization of a membrane-bound tyrosine kinase, and concentrated in and around the actin protrusion. These data show that a fraction of cells that failed to re-grow microtubules following short-term exposure to nocodazole localized NTRK2/TrkB protein to vesicles at or near an actin protrusion of the cell edge.

In summary, all 5 proteins, including 4 that associated with the ribbon-like structure, localized to cells differentially sensitive to nocodazole (TRIM29, Hs.516830, and NTRK2/TrkB in this study; LTB4DH and OKL38 are data not shown).

D. TRIM-GFP Fusion Confirmed Subcellular Localization to the Novel Ribbon-Like Structure

In an effort to confirm the subcellular localization obtained by immunofluorescence microscopy, HBEC primary cultures were infected with a retroviral construct expressing TRIM29 fused to GFP. Strong or moderate expression of GFP was observed in approximately 5% of the primary cells.

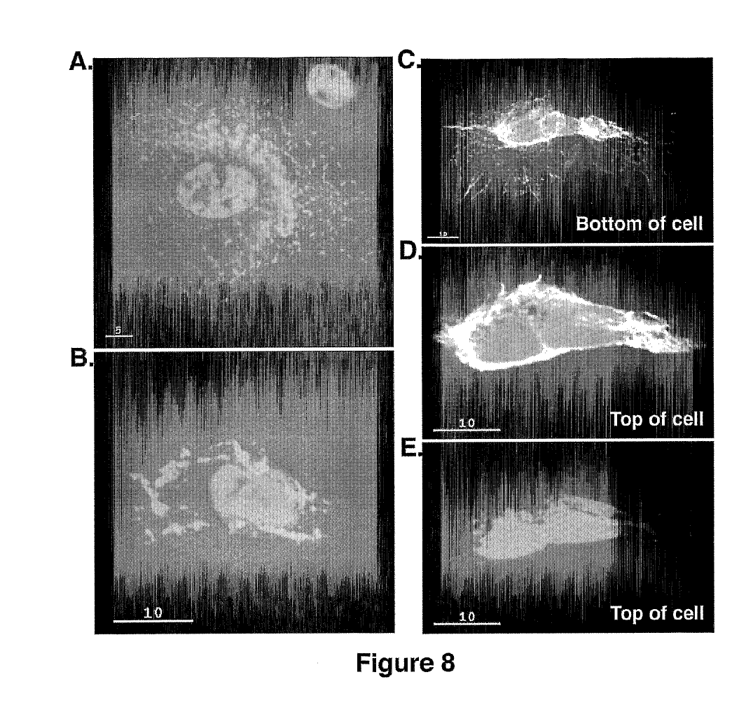

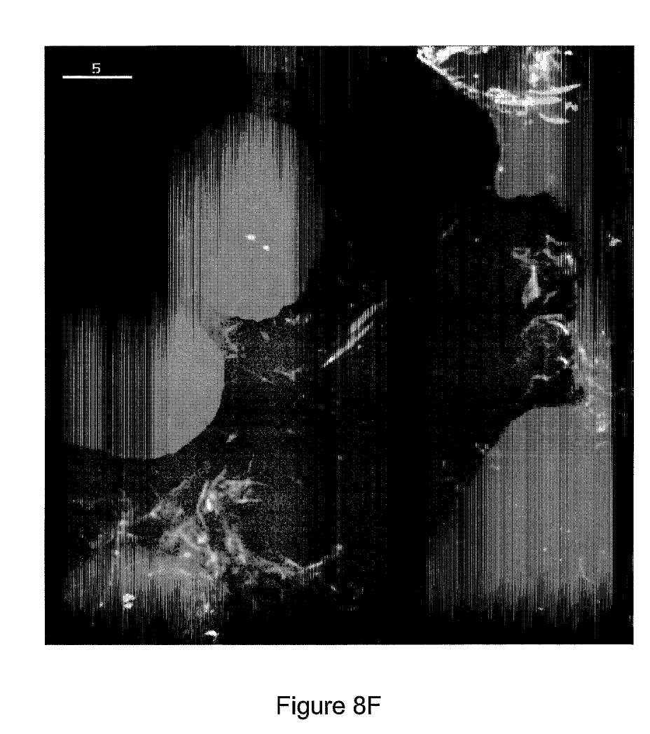

In a rare case, TRIM29-GFP localized to hundreds of tiny cylindrical structures in a single primary human bronchial epithelial cell (FIG. 8A). Preliminary evidence using live cell imaging suggested that the tiny cylindrical structures condense into a fibrous network. TRIM29-GFP localized to a fibrous network in the majority of HBEC that expressed the fusion protein (data not shown).

HBEC primary cultures also localized the TRIM29-GFP to a ribbon-like structure. The structure (green) was observed preferentially on the top of the cell (FIG. 8B) and pushed into a channel within the nucleus (blue). TRIM29-GFP did not localize significantly on the bottom of the cell and did not resemble closely the localization of actin filaments on the top of the cell (data not shown). The ribbon-like structure was also observed in a bi-nucleated cell. From the appearance of the actin cytoskeleton (red), there were possibly two cells in the process of fusion. The ribbon-like structure was observed preferentially on the top of the cell (FIG. 8 C-E, compare stacked images from bottom and top of cell) and pushed into a channel in only one of two nuclei.

In addition to primary cell cultures, we observed a ribbon-like structure in the 293T human kidney cell line. TRIM29-GFP localized to at least two linear structures (green) very near a small parcel of DNA (blue) (FIG. 8F). The actin cytoskeleton (red) implied that the parcel of DNA localized to an extension of the cell (observe cell in upper right corner) and that this parcel of DNA was well separated from the cell body and the remainder of the nucleus. This same cell also localized TRIM29 to ribbon-like structures in the vicinity of the nucleus. These data indicate that cell lines, in addition to primary cell cultures, localized TRIM29 to ribbon-like structures that are involved in moving chromosomes.

The localization of TRIM29-GFP to the ribbon-like structure was therefore entirely consistent with the results obtained above by immunofluorescence using anti-TRIM29 antiserum. TRIM29-GFP fusion experiments also provided indirect confirmation for the localization of LTB4DH, Hs.516830, and OKL38 proteins to similar sub-cellular structures by immunofluorescence microscopy.

E. Live Cell Imaging Confirmed the Presence of TRIM29-GFP in a Migrating Cell.

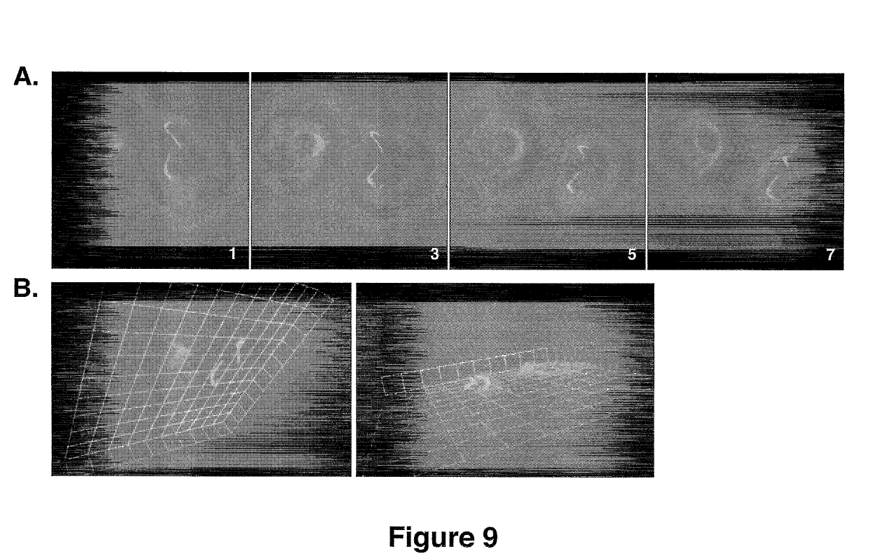

We were able to localize TRIM29-GFP in a migrating bronchial epithelial cell in real time. Using confocal microscopy, we focused on two cells in very close proximity that expressed GFP. Optical planes were stacked, and 16 time series images were inserted into movie format. Still frames that represent time points 1, 3, 5, and 7 are shown in FIG. 9A. Background GFP fluorescence was used to approximate cell borders; the lack of background fluorescence probably indicates the location of the nucleus. The morphology of the cell on the right suggests the presence of a lamellipodium and implies cell migration. We observed over the course of 1.5 hours rapid cell migration in the direction of the lamellipodium. The long axis of the cell shifted nearly 90 degrees over the time course.

TRIM29-GFP localized to two thin ribbon structures that pointed in the direction of cell migration. Optical planes for the first image in the time series were stacked, 3D rendered, and the structure was rotated in virtual space. Two different orientations are shown as a still frame in FIG. 9B. Each of the two ribbon structures bent approximately 90 degrees. The direction of the bend corresponded to the direction of cell migration. We also observed that both ribbon structures ran parallel with each other and were qualitatively identical.

F. Genes Encoding LTB4DH, OKL38, Hs.516830, and NTRK2/TrkB Proteins were Expressed in Squamous Lung Tumors.

We analyzed by cDNA microarray analysis the global gene expression patterns from resected human tumors of squamous differentiation using 42,000 clone cDNA microarrays representing 26000 human genes. We included in the analysis 40 patients with SCC of the lung, 27 patients with SCC of the head/neck, and three normal lung tissues.

We focused our analysis on 2150 DNA clones representing approximately 1600 unique genes based on two criteria. First, repeated sampling for 6 lung and 5 head/neck tumors was used to select genes that were well measured. Second, the expression pattern for each gene varied widely across the 11 tumor pairs. These 1600 genes best represent the diversity of gene expression in SCC.

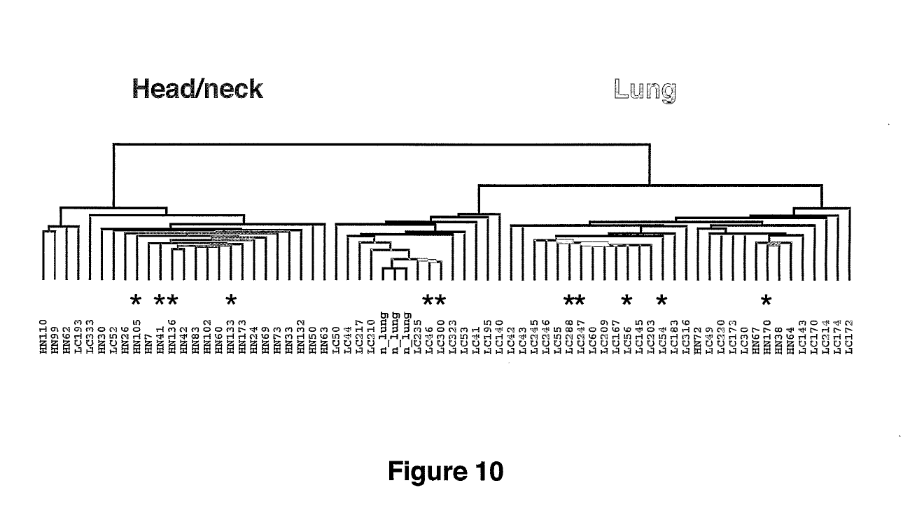

Hierarchical cluster analysis was used to sort 2150 clones and 70 tissues based on similarities in gene expression. The diverse patterns of gene expression for the two morphologically indistinguishable squamous tumors were visualized using TreeView. With a few notable exceptions, the gene expression patterns largely distinguished lung (orange) and head/neck (blue) SCC, as indicated by the colored branches of the dendogram shown in FIG. 10.

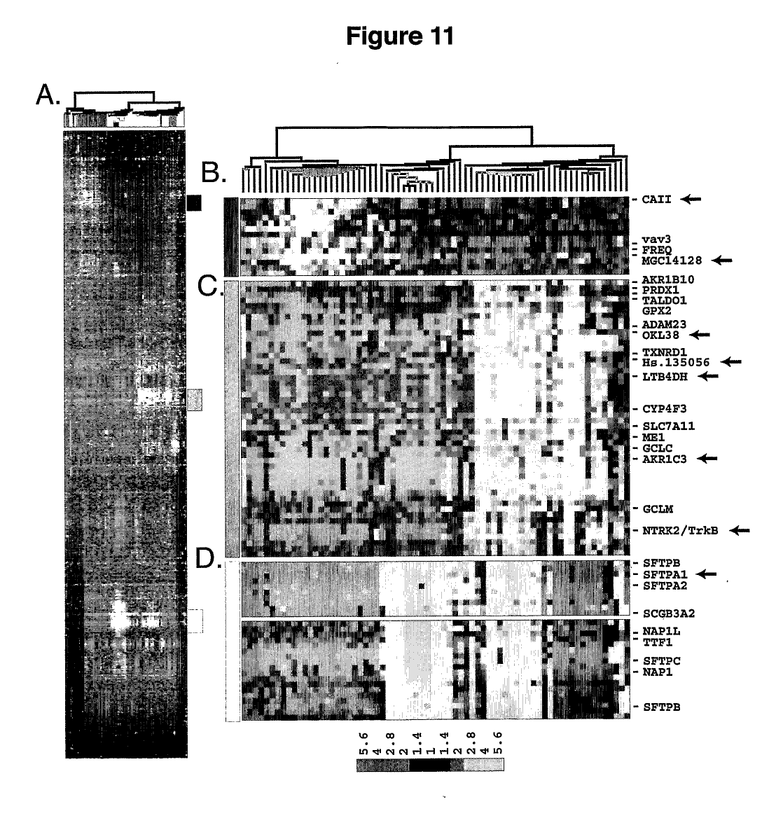

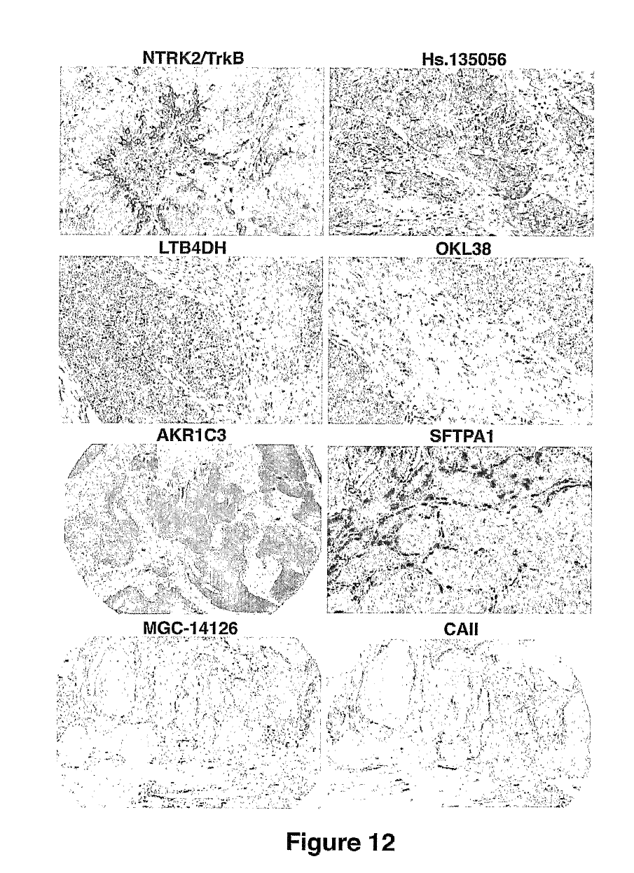

The entire gene cluster containing 2150 clones and 70 tissues is shown in FIG. 11A. We observed three dominant gene expression patterns, which were expanded on the right to include gene names. Arrows to the right of gene names indicate that the presence of the gene or gene product in tumor cells was confirmed by in situ hybridization or immunohistochemistry (see FIG. 12 below).

Gene expression pattern 1 was strongly expressed in 19 of 27 head/neck tumors and two lung tumors (FIG. 11B). There were 8 SCC of the head/neck that expressed these genes poorly. Although the distinction is not absolute, gene expression pattern 1 largely represented head/neck SCC and was poorly expressed in lung SCC. Gene expression pattern 2 was expressed strongly in 26 out of 40 lung and 5 head/neck squamous tumors (FIG. 11C). There were 14 SCC of the lung that expressed these genes poorly. Pattern 2 largely represented lung SCC and was poorly expressed in SCC of the head/neck, although, like pattern 1, the distinction was not absolute.

A third, very prominent gene expression pattern found only in lung SCC contained several genes expressed normally in non-tumor lung tissue, including, among others, surfactant, napsin, and TTF1. As expected, this expression pattern was shared with that from normal lung (FIG. 11D). The strong gene expression pattern containing surfactant, napsin, and TTF1 genes was most likely the result of residual normal lung and does not necessarily represent diversity of gene expression in SCC of the lung (see FIG. 12 below).

Gene expression pattern 2, which was differentially expressed in the majority of lung SCC, contained numerous genes that encode proteins with intrinsic enzymatic activity. Several of these genes are shown in FIG. 11C. A more extensive list of genes that correlated strongly with leukotriene B4 12-hydroxydehydrogenase (LTB4DH) is shown in Table 3, above.

Gene expression pattern 2 was highly enriched in oxidoreductases that utilize or regenerate NADPH. As an example, NADPH-dependent LTB4DH and cytochrome P450 F3 (CYP4F3) both metabolize the inflammatory lipid LTB4. Genes involved in glutathione homeostasis included cystine/glutamate transporter (SLC7A11), glutathione reductase, and glutamate-cysteine ligase catalytic (GCLC) and modifier (GCLM) subunits. Malic enzyme 1 (ME1) and three key genes in the pentose phosphate pathway, transaldolase (TALDO1), transketolase, and phosphogluconate dehydrogenase, generate NADPH for fatty acid biosynthesis. Peroxiredoxin 1, thioredoxin 1 (TXNRD1), and glutathione peroxidase 2 (GPX2) contribute to redox homeostasis. Peroxiredoxin 1 localizes to the mitochondria.

We observed several genes with unknown function in gene expression pattern 2. These included the oxidoreductase OKL38, aldo-keto reductase (AKR), Hs.516830 containing a putative nuclease domain, and neurotrophic tyrosine kinase receptor type 2 (NTKR2/TrkB), whose function in non-neuronal tissues is largely unknown.

Immunohistochemistry or in situ hybridization on formalin-fixed, paraffin-embedded human tumors was used to determine whether select genes were expressed in squamous tumor cells. TrkB, Hs.516830, AKR1C3, LTB4DH, and OKL38 were selected from gene expression pattern 2. CAII and the hypothetical protein MGC-14128 (accession BC007828) were taken from gene expression pattern 1.

Immunohistochemistry and affinity-purified polyclonal antisera showed that TrkB, Hs.516830, and AKR1C3 proteins strongly and specifically localized to the tumor cells in SCC of the lung (FIG. 12).

In situ hybridization localized LTB4DH and OKL38 mRNAs in tumor cells from SCC of the lung (FIG. 12). Similarly, CAII and MGC-14128 mRNAs localized specifically to tumor cells from SCC of the head/neck (FIG. 12). In situ hybridization revealed the presence of surfactant A1 (SFTPA1) message in a cell type consistent with “normal” type II pneumocytes (FIG. 12). As expected, surfactant mRNA was not found in squamous tumor cells.

G. TRIM29 Protein Expression in Tumor Tissue Predicted Poor Prognosis for Human Adenocarcinoma of the Lung.

Our gene expression studies showed that TRIM29 mRNA correlated with poor prognosis for patients with adenocarcinoma of the lung. Immunohistochemistry and archival, paraffin-embedded tumors were used to determine whether the gene product for TRIM29 predicts patient outcomes in 39 patients with lung adenocarcinoma.

Polyclonal antisera to TRIM29 was peptide affinity-purified. Characterization of TRIM29 antiserum was described in FIG. 2 above.

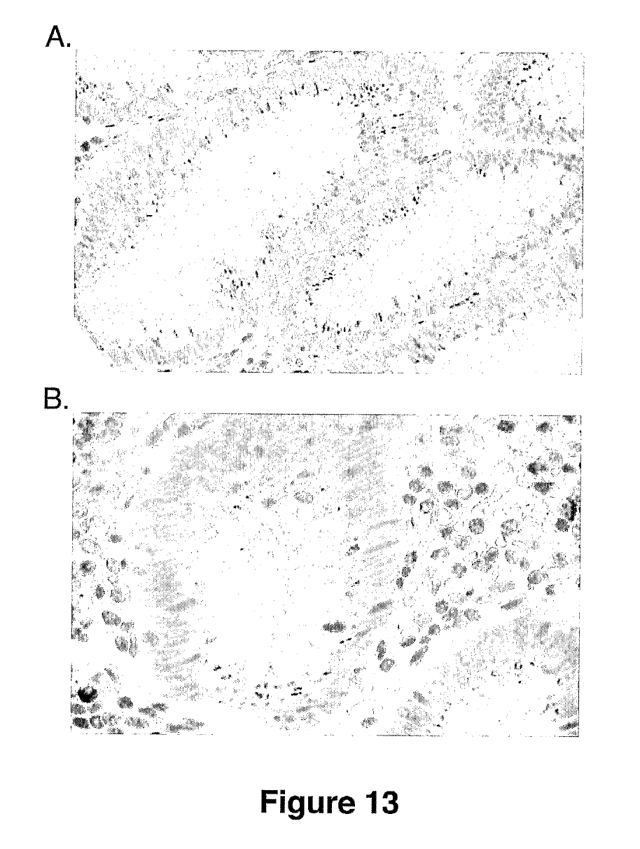

Immunohistochemical staining across a panel of paraffin-embedded normal human tissues showed a punctate, apical localization for TRIM29 protein in colon adenoma (FIG. 13A) and normal appendix (FIG. 13B).

Formalin-fixed, paraffin-embedded archival tumors from 39 patients with adenocarcinoma of the lung were obtained from Vancouver General Hospital. Morphological diagnosis and clinical follow-up for each patient are shown in Table 4. The clinical records showed that 19 of the 39 patients died from the tumor.

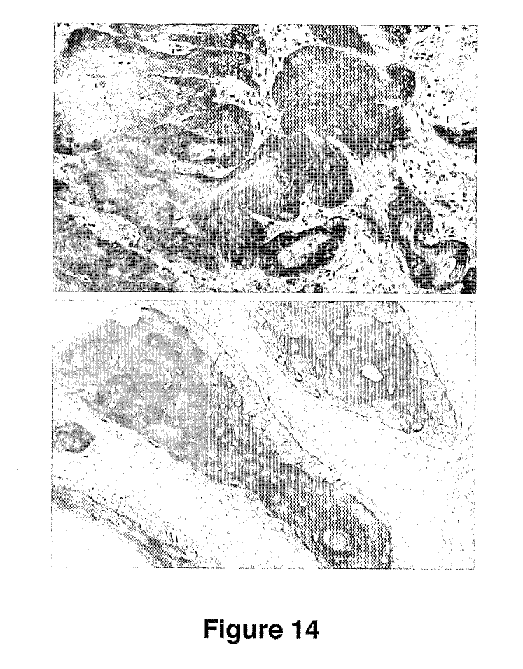

Immunohistochemistry showed that TRIM29 (FIG. 14A) was expressed in the tumor cells from lung adenocarcinomas. TRIM29 protein was strongly expressed in SCC of the head/neck (FIG. 14B) and lung (data not shown). These results are consistent with previous gene expression studies that showed strong mRNA expression for TRIM29 in SCC of the lung and a subset of lung adenocarcinomas (Garber et al., 2001), and provide evidence to suggest that the TRIM29 antiserum was specific for the intended protein.

Results for the immunohistochemical stains are listed in Table 4.

TABLE 4

| tumor | | | | | stage | last | | | | |
|---|---|---|---|---|---|---|---|---|---|---|
| ID | TRIM29 | SLC7A5 | Sex | age | UICC | follow-up | tofudays | tofuyear | Status | morphological diagnosis |
| 136 | 1 | 1 | F | 67 | 1 | 1 Jan. 2002 | 253 | 0.693151 | 1 | moderately differentiated aden [symbol] |
| 138 | 1 | 1 | M | 70 | 1 | 1 Jan. 2002 | 132 | 0.361644 | 1 | moderately differentiated aden [symbol] |
| 139 | 1 | 1 | F | 57 | 1 | 1 Jan. 2002 | 105 | 0.287671 | 1 | moderately differentiated non-n [symbol] |
| 135 | 1 | 1 | F | 82 | 1 | 1 Jan. 2002 | 308 | 0.843836 | 1 | moderately differentiated aden [symbol] |
| 124 | 1 | 1 | F | 71 | 1 | 7 Apr. 1987 | 1086 | 2.975342 | 4 | poorly differentiated adeno |
| 117 | 2 | 2 | M | 66 | 3 | 2 Jul. 1986 | 646 | 1.769863 | 4 | adenosquamous |
| 118 | 1 | 1 | M | 56 | 1 | 15 Nov. 1985 | 248 | 0.679452 | 5 | bronchoalveolar |
| 120 | 3 | 1 | M | 77 | 4 | 6 Nov. 1986 | 184 | 0.50411 | 6 | poorly differentiated adenosqu [symbol] |
| 108 | 3 | 3 | F | 74 | 4 | 12 Oct. 1987 | 94 | 0.257534 | 4 | mucinous bronchoalveolar |
| 109 | 1 | 1 | F | 61 | 1 | 1 Jan. 2002 | 4900 | 13.42466 | 1 | bronchoalveolar |
| 112 | 1 | 1 | M | 75 | unknown | 24 Feb. 1993 | 1553 | 4.254795 | 4 | bronchoalveolar |
| 110 | 1 | 3+ | F | 71 | 1 | 3 Oct. 1993 | 1984 | 5.435616 | 6 | moderately differentiated aden [symbol] |
| 111 | 3 | 1 | F | 38 | 3 | 11 Feb. 1989 | 236 | 0.646575 | 4 | bronchoalveolar |
| 123 | 1 | 1 | M | 50 | 1 | 15 Oct. 1992 | 1050 | 2.876712 | 4 | poorly differentiated adeno |
| 122 | 1 | 1 | M | 77 | 1 | 8 Nov. 1993 | 844 | 2.312329 | 4 | moderately differentiated aden [symbol] |
| 113 | 2 | 3 | F | 63 | 1 | 12 Jan. 1992 | 248 | 0.679452 | 4 | mucinous adeno |
| 90 | 1 | 1 | F | 74 | lung met | 23 Nov. 1994 | 107 | 0.293151 | 4 | adeno with papillary pattern |
| | | | | | to brain | | | | | |
| 125 | 1 | 1 | F | 70 | 1 | 1 Jan. 2002 | 2513 | 6.884932 | 1 | poorly differentiated adeno |
| 91 | 3+ | 3 | F | 76 | 2 | 27 May 1997 | 833 | 2.282192 | 4 | adenosquamous |
| 92 | 3 | 3+ | M | 75 | 1 | 13 May 1995 | 74 | 0.20274 | 4 | bronchioloalveolar |
| 93 | 1 | 1 | M | 55 | 1 | 2 Sep. 1995 | 186 | 0.509589 | 6 | well to moderately differentiate [symbol] |
| 94 | 1 | 1 | M | 66 | 2 | 13 Feb. 1998 | 956 | 2.619178 | 4 | adeno |
| 95 | 1 | 1 | M | 62 | 1 | 18 Apr. 1998 | 1115 | 3.054795 | 4 | bronchioalveolar |
| 96 | 1 | 1 | F | 74 | 1 | 1 Jan. 2002 | 2340 | 6.410959 | 1 | moderately differentiated aden [symbol] |
| 97 | 1 | 1 | F | 67 | 1 | 17 Nov. 1995 | 157 | 0.430137 | 4 | moderately differentiated aden [symbol] |
| 98 | 1 | 3+ | M | 80 | 3 | 27 Jan. 1998 | 950 | 2.60274 | 4 | poorly differentiated adeno |
| 99 | 1 | 1 | F | 52 | 4 | 1 Jan. 2002 | 2262 | 6.19726 | 1 | well differentiated bronchioalve [symbol] |
| 121 | 1 | 3 | M | 65 | 1 | 1 Jan. 2002 | 2239 | 6.134247 | 1 | well to moderately well differen [symbol] |
| 100 | 1 | 3 | F | 64 | 1 | 1 Jan. 2002 | 2234 | 6.120548 | 1 | bronchioloalveolar |
| 101 | 1 | 1 | F | 62 | 1 | 21 Jan. 1997 | 173 | 0.473973 | 4 | bronchoalveolar |
| 102 | 1 | 1 | F | 72 | 1 | 1 Jan. 2002 | 2045 | 5.60274 | 1 | bronchioloalveolar |
| 104 | 1 | 1 | M | 65 | 1 | 1 Jan. 2002 | 2183 | 5.980822 | 1 | bronchoalveolar |
| 106 | 1 | 1 | F | 63 | 1 | 10 Sep. 2000 | 1136 | 3.112329 | 4 | mucinous adeno |
| 107 | 1 | 1 | M | 79 | 1 | 1 Jan. 2002 | 1704 | 4.668493 | 1 | mucinous adeno |
| 126 | 1 | 1 | F | 52 | 1 | 18 Apr. 2001 | 1115 | 3.054795 | 6 | poorly differentiated adeno |
| 129 | 1 | 3 | F | 56 | 2 | 1 Jul. 1999 | 309 | 0.846575 | 4 | moderately differentiated aden [symbol] |
| 131 | 1 | 1 | M | 74 | 1 | 1 Jan. 2002 | 977 | 2.676712 | 1 | moderately differentiated aden [symbol] |
| 132 | 1 | 3 | F | 56 | 3 | 3 Jun. 2001 | 734 | 2.010959 | 4 | poorly differentiated adeno |
| 130 | 1 | 1 | M | 48 | 3 | 1 Jan. 2002 | 1071 | 2.934247 | 1 | acinar adeno/bronchioloalveola [symbol] |

IHC scores. 1: negative; 2: moderate, 3: strong
yellow bar indicates staining for all three antisera
plus sign indicates very strong expression

A strong or weak immunostain was considered positive. TRIM29 stained 7 adenocarcinomas, or 18% of the 39 adenocarcinoma lung tumors examined in this study. 6 of 7 patients that stained with TRIM29 died from the tumor. Patients were segregated into two groups based on immunohistochemical stains. A positive stain represented one group; no stain represented the second group. An event refers to the number of patients in each group that died from the tumor. Kaplan-Meier analysis showed that the expression of TRIM29 protein in lung adenocarcinomas correlated with poor prognosis, with a p value less than 0.00001.

H. The Ribbon-Like Structure Localized in the Vicinity of Condensed, Mitotic Chromosomes in Cells Treated with Paclitaxel

In an attempt to examine further the contribution of microtubules to the ribbon-like structure, we treated HBEC with the microtubule-stabilizing drug paclitaxel (commonly referred to as taxol). Paclitaxel, used clinically to treat several human cancers, blocks tumor cell division apparently by inhibiting the mitotic spindle.

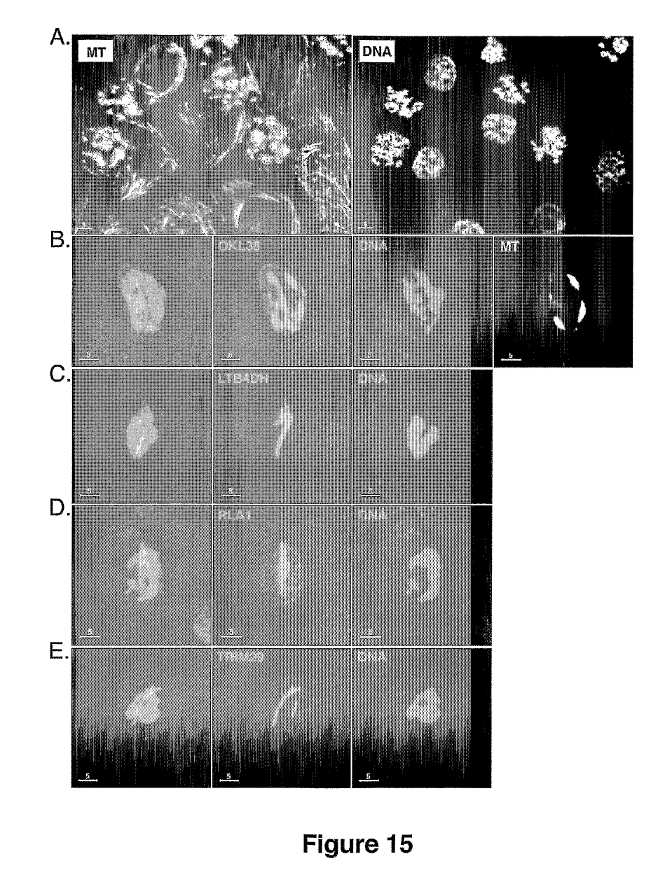

In HBEC treated with paclitaxel for 9 hours, we observed a 10-fold increase in the number of mitotic figures based on DAPI stain of fixed cells. Mitotic figures were evenly spread across the culture. Immunofluorescence microscopy showed that alpha-tubulin localized to intense foci in HBEC with condensed, seemingly mitotic DNA (FIG. 15A). Such foci were not observed in the absence of paclitaxel. Based on microtubule localization, there was no indication of an intact mitotic spindle in any cell.

OKL38, LTB4DH, RLA1, and TRIM29 localized to ribbon-like structures in HBEC treated with paclitaxel (FIG. 15B-E) with several notable characteristics. First, localization was restricted to cells on the periphery of the culture, consistent with all previous data on the localization of the ribbon-like structure in HBEC culture. Second, the four proteins localized to a ribbon-like structure in mitotic cells with condensed DNA and intense microtubule foci. We did not observe the ribbon-like structure in cells without mitotic, condensed DNA. Third, HBEC treated with paclitaxel localized a ribbon-like structure in close proximity to well organized, mitotic DNA. In the case of OKL38 (FIG. 15B, green) and LTB4DH (FIG. 15C, green), mitotic DNA was ordered on either side of the ribbon-like structure (compare DAPI stain between FIG. 15A and FIG. 15B-E). For Hs.516830 (RLA1), mitotic DNA was aligned and parallel with the ribbon-like structure in a manner reminiscent of metaphase chromosomes during normal cell division (FIG. 15D). Fourth, OKL38 and microtubules showed striking co-localization to foci adjacent to mitotic DNA (FIG. 15B). Consistent with previous findings, OKL38 but not microtubules localized to the ribbon-like structure (FIG. 15B).

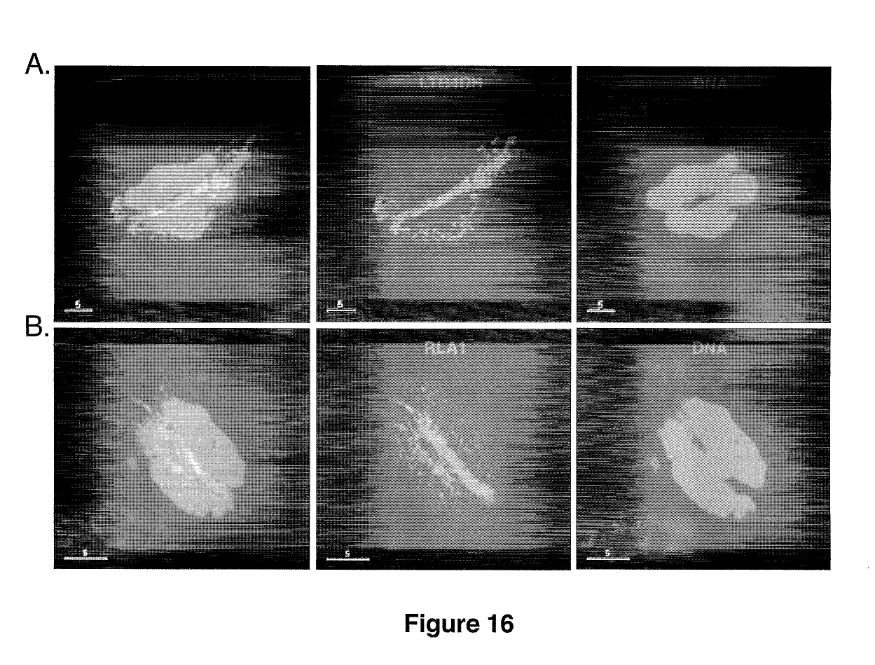

The localization of a ribbon-like structure in the vicinity of condensed, mitotic DNA in cells treated with paclitaxel was not specific to primary lung HBEC. We observed a very similar phenomenon with the immortal breast epithelial cell line MCF10A. LTB4DH (FIG. 16A, green) and OKL38 (FIG. 16B, green) localized in the vicinity of condensed, mitotic DNA (blue) in MCF10A cells treated with paclitaxel.

Together, these data indicate that paclitaxel promotes the formation of a ribbon-like structure in close proximity to mitotic DNA. This is in contrast to cells in the absence of paclitaxel, where antisera to the four proteins did not localize to a ribbon-like structure in mitotic cells.

In order to confirm the subcellular localization obtained by immunofluorescence microscopy and to provide independent evidence as to the specificity of the antisera, we stably expressed TRIM29 fused to GFP in an effort to determine whether the GFP fusion protein co-localized with anti-TRIM29 antiserum to the ribbon-like structure. For these experiments, we used the immortal breast epithelial cell line MCF10A.

TRIM29-GFP (FIG. 17A, left) and anti-TRIM29 (FIG. 17A, right) co-localized to a ribbon-like structure in cells treated with paclitaxel. Two additional examples that show identical results are given for comparison (FIGS. 17B and 17C). The localization of TRIM29-GFP to the ribbon-like structure was therefore entirely consistent with the results obtained by immunofluorescence using anti-TRIM29 antiserum. These results provide additional evidence to suggest that the TRIM29 antibody was specific for the TRIM29 protein.

In primary cells treated with the microtubule-stabilizing drug paclitaxel, we observed an abnormal mitotic spindle in every mitotic cell. The ribbon-like structure was not observed in every mitotic cell. To the contrary, it was observed in select cells on the periphery of the culture, consistent with the localization of this structure in select cells differentially sensitive to nocodazole. In cells treated with paclitaxel, the ribbon-like structure localized in the vicinity of condensed, mitotic chromosomes, which was not observed in the absence of paclitaxel or following recovery from nocodazole.

This study shows that four proteins (TRIM29, RLA1 (Hs.516830), OKL38, and LTB4DH) and the ribbon-like structure localized very near condensed, mitotic chromosomes in cells treated with paclitaxel. The above data show that paclitaxel promotes the formation of a ribbon-like structure in close proximity to mitotic DNA. Since the clinical benefit of paclitaxel results from the detrimental effects associated with a dysfunctional mitotic spindle in tumor cells, the above results show that the ribbon-like structure contributes cytofilaments, similar to the mitotic spindle, that function as a primitive mitotic spindle to restore genomic integrity. Such a scenario bypasses the killing effect of a mitotic spindle inhibitor and contributes to drug resistance, a common problem associated with the clinical use of paclitaxel.

Therefore, administration of an inhibitor of one or more of the enzymes associated with the ribbon-like structure in conjunction with paclitaxel or a functional analogue thereof will improve the effectiveness of paclitaxel, in certain embodiments in a synergistic fashion, e.g., by reducing the occurrence of paclitaxel resistance.

I. Hs.516830 (RLA1) Co-Localized with Mitochondria in Breast MCF10A Cells.

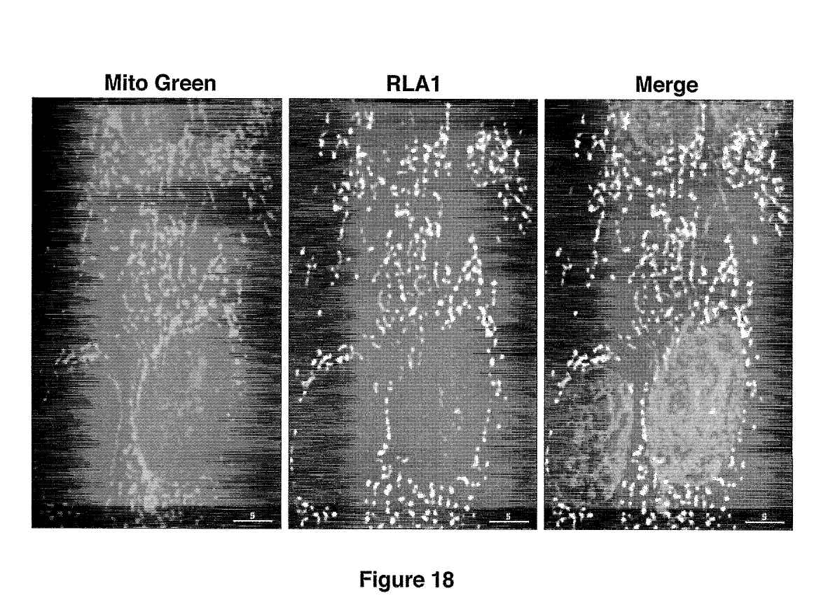

In addition to its localization on the ribbon-like structure, RLA1 (Hs.516830) also localized to distinct, small foci in the breast epithelial cell line MCF10A (FIG. 18). Using MitoTracker Green, a small molecule shown to localize to mitochondria in formaldehyde fixed cells, we observed that antiserum directed against the Hs.516830 protein co-localized with the mitochondrial stain. These results indicated that Hs. 516830 protein localized to mitochondria in MCF10A cells. We also observed similar localization for Hs.516830 in primary HBEC (data not shown).

Hs.516830 was one of many genes that strongly correlated with LTB4DH mRNA expression in human squamous tumors using cDNA microarray analysis (see Table 3). These genes were highly enriched in oxidoreductases that utilize or regenerate NADPH. It is interesting to note that one of the many functions associated with the mitochondria is to regulate cytosolic NADPH. The localization of Hs.516830 to the mitochondria is consistent with a role for these genes in mitochondrial function. The above findings indicate that genes contained within Table 3 contribute, directly or indirectly, to the function of Hs.516830. Modulating the activity or level of the genes listed in Table 3 therefore may be used in certain embodiments to modulate the activity of Hs.516830.

III. Discussion

The above results demonstrate that TRIM29, Hs.516830, LTB4DH, and OKL38 localized specifically to a novel, ribbon-like structure in a minority of cells in culture. The ribbon-like structure pushed through the nucleus, creating a nuclear channel. Localization of LTB4DH to the ribbon-like structure was largely confined to the nuclear boundary, whereas Hs.516830 and OKL38 showed a more extensive structure that extended beyond the nucleus. Interestingly, TRIM29, Hs.516830, OKL38, LTB4DH, and NTKR2/TrkB localized to the ribbon-like structure in a minority of primary cells in culture that were differentially sensitive to nocodazole. Localization of TRIM29 to the ribbon-like structure was confirmed in primary lung epithelial cells and in a breast epithelial cell line using a TRIM29-GFP fusion protein.

Both TRIM29 and NTRK2/TrkB proteins localized to discrete structures in cells that implied an active cell migration. In these cells, the actin cytoskeleton showed a lamellipodial-like protrusion. Based on cell culture models, lamellipodia actively participate in cell migration. NTRK2/TrkB localized to vesicles very near the site of actin protrusion. Live cell imaging confirmed the presence of TRIM29-GFP bound to a ribbon-like structure in a migrating epithelial cell.

A. A Hypothetical Model for the Five Proteins in Tumor Progression

The above described subcellular localization for the five gene products evaluated in this study shows that these five gene products function as a cohesive unit. Though not wishing to be bound to any particular theory, we suggest two possible mechanisms of how these 5 proteins may contribute to tumor progression. First, NTRK2/TrkB protein concentrates around the leading edge extension, suggesting that this tyrosine kinase may have a role in the regulation of the leading edge. As plasma membranes extend and retract in a migrating cell, mechanisms must also exist to move internal organelles, such as the nucleus. Nucleokinesis refers to the process of active nuclear positioning during cell movement. We discovered four proteins that may contribute to nucleokinesis. These 4 proteins localize to a ribbon-like structure that pushes through the nucleus and guides the nucleus down a localized track in the direction of cell movement.

Second, previous studies have shown that OKL38 and TRIM29 proteins contribute to DNA repair and genome stability (see section II Results). RLA1 (Hs.516830) binds DNA and contributes directly to segregation of bacterial genomes during cell division. Together, the implied function for these three genes is to maintain genome stability. These prior data are consistent with a direct role for these 3 proteins in countering the detrimental and often toxic effects of paclitaxel on the mammalian genome. It is also consistent with the subcellular localization for the 3 proteins in the vicinity of the DNA.

B. The Ribbon-Like Structure is a Highly Enzymatic Process

Four of the five proteins characterized in this study are putative enzymes. Three enzymes localized to the ribbon-like structure, including Hs.516830, LTB4DH, and OKL38. TRIM29 was the only protein that had no known intrinsic enzymatic activity.

C. Proteins Associated with the Ribbon-Like Structure were Expressed in Lung Tumors and Predicted Poor Prognosis for Adenocarcinoma of the Lung

We showed that TRIM29 protein was preferentially expressed in select normal human tissues and in adenocarcinoma lung tumor cells. TRIM29 accurately identified a subset of patients with poor prognosis for adenocarcinoma of the lung.

TRIM29, Hs.516830, and NTRK2/TrkB proteins were strongly expressed in SCC of the lung. We have also showed that OKL38 and LTB4DH mRNAs were expressed in squamous cell carcinoma of the lung.

It is evident from the above results and discussion that the subject invention provides new methods of diagnosing and treating neoplastic disease conditions, such as lung cancer. Accordingly, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of evaluating a cell's metastatic propensity, said method comprising:
assaying said cell for the presence of at least one target protein associated with cellular locomotion to obtain a result; and
using said result to evaluate said cell's metastatic propensity.

2. The method according to claim 1, wherein said at least one target protein is a nucleus-associated ribbon-like structure protein.

3. The method according to claim 1, wherein said nucleus-associated ribbon-like structure protein is chosen from:
Leukotriene B4 12-hydroxydehydrogenase (LTB4DH);
Pregnancy-induced growth inhibitor (OKL38);
Hs.516830 (C20orf139);
cyp4 proteins; and
Tripartite-containing motif 29 (TRIM29).

4. The method according to claim 1, wherein said at least one target protein is a leading edge cellular locomotion protein.

5. The method according to claim 4, wherein said leading edge cellular locomotion protein is Neurotrophic tyrosine kinase receptor type 2 (NTRK2/TrkB).

6. The method according to claim 1, wherein said assaying comprises assaying said cell for the presence of at least two different target proteins in said cell.

7. The method according to claim 1, wherein said assay comprises assaying said cell for the presence of a nucleus-associated ribbon-like structure.

8. The method according to claim 7, wherein said nucleus-associated ribbon-like structure comprises:
Leukotriene B4 12-hydroxydehydrogenase (LTB4DH);
Pregnancy-induced growth inhibitor (OKL38);
Hs.516830 (C20orf139);
cyp4 proteins; and
Tripartite-containing motif 29 (TRIM29).

9. The method according to claim 1, wherein said cell is a neoplastic cell.

10. The method according to claim 9, wherein said neoplastic cell is a tumor cell.

11. The method according to claim 10, wherein said tumor cell is from a tumor harvested from a subject suffering from a neoplastic disease.

12. The method according to claim 11, wherein said neoplastic disease is a lung cancer.

13. The method according to claim 12, wherein said lung cancer is adenocarcinoma.

14. A method of making a prognosis for a subject suffering from a neoplastic disease, said method comprising:
assaying a cell obtained from said subject for the presence of at least one target protein associated with cellular locomotion to obtain a result; and
using said result to make a prognosis for said subject.

15. The method according to claim 14, wherein said at least one target protein is a nucleus-associated ribbon-like structure protein.

16. The method according to claim 15, wherein said nucleus-associated ribbon-like structure protein is chosen from:
Leukotriene B4 12-hydroxydehydrogenase (LTB4DH);
Pregnancy-induced growth inhibitor (OKL38);
Hs.516830 (C20orf139);
cyp4 proteins; and
Tripartite-containing motif 29 (TRIM29).

17-54. (canceled)

55. A kit for use in evaluating a cell's metastatic propensity, said kit comprising:
a reagent for assaying a cell for the presence of at least one target protein associated with cellular locomotion.

56. The kit according to claim 55, wherein said at least one target protein is a nucleus-associated ribbon-like structure protein.

57. The kit according to claim 56 wherein said nucleus-associated ribbon-like structure protein is chosen from:
Leukotriene B4 12-hydroxydehydrogenase (LTB4DH);
Pregnancy-induced growth inhibitor (OKL38);
Hs.516830 (C20orf139);
cyp4 proteins; and
Tripartite-containing motif 29 (TRIM29).

58-80. (canceled)
